(19) United States
(12) Patent Application Publication
(10) Pub. No.: US 2008/0182328 A1
(43) Pub. Date: Jul. 31, 2008

(54) Mammalian extraembryonic endoderm cells and methods of isolation

(75) Inventors: Evan Y. Snyder, La Jolla, CA (US); Rodolfo Gonzalez, La Jolla, CA (US); Jeanne F. Loring, La Jolla, CA (US); Prithi Rajan, La Jolla, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Burnham Institute, La Jolla, CA (US)

(21) Appl. No.: 12/004,299

(22) Filed: Dec. 19, 2007

Related U.S. Application Data
(60) Provisional application No. 60/876,004, filed on Dec. 19, 2006.

(51) Int. Cl. C12N 5/00; C12N 5/02

(52) U.S. Cl. 435/353; 435/363; 435/404; 435/372.3; 435/395; 435/354

(57) ABSTRACT

An isolated mammalian extraembryonic endoderm-like cell line is provided. Methods for producing isolated mammalian extraembryonic endoderm-like cell line derived from a mammalian pluripotent stem cell culture are provided. Primate or human embryonic stem cells (ESCs) spontaneously generate the primate or human extraembryonic endoderm-like cell line wherein the extraembryonic endoderm-like cells sustain the pluripotence of the primate or human ESCs.

### CROSS REFERENCE TO RELATED APPLICATIONS

This application claims priority to U.S. Provisional Application No. 60/876,004 filed on Dec. 19, 2006 the contents of which are expressly incorporated by reference herein in its entirety.

### STATEMENT OF GOVERNMENT SUPPORT

This invention was made with Government support of grant numbers NIH T32CA77109, NIMH 5-73177, NIAID 5-75071, YRC 5-75142, NIH P20 GM075059, NIH P20 GM075059, and NIH R01 NS040822 from the National Institutes of Health, and USDA 5-73194 from the United States Department of Agriculture. The Government has certain rights in this invention.

### FIELD

The invention relates generally to an isolated mammalian extraembryonic endoderm-like cell line. Methods for producing an isolated mammalian extraembryonic endoderm-like cell line derived from a mammalian pluripotent stem cell culture are provided. Primate or human embryonic stem cells (ESCs) spontaneously generate the primate or human extraembryonic endoderm-like cell line wherein the extraembryonic endoderm-like cells sustain the pluripotence of the primate or human ESCs. The invention further relates to methods for culturing undifferentiated human embryonic stem cells in the presence of extraembryonic endoderm-like cells in a nutrient medium. Methods for generating isolated extraembryonic endoderm-like cells are provided which comprises growing primate embryonic stem cells on extracellular matrix under feeder cell free conditions, and identifying and isolating the extraembryonic endoderm-like cells in the cell culture.

### BACKGROUND

One of the first lineages to emerge in embryogenesis is the extraembryonic primitive endoderm (PE), a transient cell population which arises at the blastocyst stage, segregates from the inner cell mass (ICM), and forms a polarized epithelial layer on the blastocoelic surface of the ICM. Rossant, Semin Cell Dev Biol. 15:573, 2004. Cells of the ICM express pluripotence-associated genes, including the transcription factor POU5F1/OCT4, while the onset of differentiation of PE cells is marked by profound changes such as the onset of expression of the transcription factor GATA-6 (Rossant and Yamanaka, Philos Trans R Soc Lond B Biol Sci. 358:1341, 2003), and extracellular matrix components. Kunath et al., Development 132:1649, 2005. The PE plays important roles in embryonic development, first by sustaining the ICM as it forms the embryonic epiblast, and second by giving rise to the extraembryonic endoderm, which is vital to nutrient transport and regulation of pattern formation during early embryogenesis. Bielinska et al., Int J Dev Biol. 43:183, 1999.

Human embryonic stem cells (hESCs) are derived from and resemble the ICM of blastocyst-stage embryos, and they are conventionally maintained in a pluripotent state by co-culture with feeder layers of mouse or human fibroblasts. A variety of fibroblastic types have been used as feeder layers, including the heterogeneous population of cells derived from mouse embryos (mouse embryo fibroblasts [MEFs]), established mouse cell lines such as STO fibroblasts, and primary populations of human fibroblasts. The common factors that allow these diverse cell types to support hESC pluripotence are unknown, but recent observations suggest that these various fibroblasts can resemble a single cell type that serves a similar function during embryonic development in vivo. When cultured in the absence of a fibroblast feeder layer, hESCs spontaneously giving rise to subpopulations of cells that migrate out from the hESC colonies. Xu et al., Nat Biotechnol 19:971, 2001; Rosler et al., Dev Dyn 229:259, 2004. As these early-differentiating cell types emerge from individual hESC colonies, the remaining hESCs within the colonies continue to proliferate and remain pluripotent, suggesting that the hESC derivatives can function in a similar fashion to the feeder layers that are routinely used to maintain hESC in an undifferentiated state. A need exists in the art for improved in vitro cell culture conditions for the isolation and propagation of embryonic stem cells.

### SUMMARY

The present invention provides an isolated mammalian extraembryonic endoderm-like cell line or variant cell line thereof. Methods for producing an isolated mammalian extraembryonic endoderm-like cell line derived from a mammalian pluripotent stem cell culture are provided. The cells can be obtained from a mammal, including but not limited to, primate, human, rat or mouse. Primate or human embryonic stem cells (ESCs) spontaneously generate the primate or human extraembryonic endoderm-like cells wherein the extraembryonic endoderm-like cells sustain the pluripotence of the primate or human ESCs. The extraembryonic endoderm-like cell line comprises a gene and protein expression profile having decreased expression of genes associated with undifferentiated human embryonic stem cells and increased expression of genes associated with extraembryonic endoderm, e.g., primitive endoderm, parietal endoderm, or visceral endoderm. A conditioned medium from the primate extraembryonic endoderm-like cells provides factors for the growth and maintenance of primate or human ESCs.

A method for culturing undifferentiated human embryonic stem cells, is provided which comprises obtaining a single undifferentiated human embryonic stem cell, and inoculating the single cell onto extraembryonic endoderm-like feeder cells in a nutrient medium. A method for generating isolated extraembryonic endoderm-like cells is provided which comprises growing primate embryonic stem cells on extracellular matrix under feeder cell free conditions; identifying extraembryonic endoderm-like cells as positive for cell markers of one or more of the following: GATA6, DAB-2, basement membrane genes, laminin (LAMC1), collagens, fibronectin (FN1), or nidogens; and isolating extraembryonic endoderm-like cells from the embryonic stem cells.

Human embryonic stem cells (hESCs), which are derived from the ICM of blastocyst-stage embryos, often generate a subpopulation of fibroblast-like cells when they are cultured in the absence of a feeder layer. As this early-differentiating cell type emerges from individual hESC colonies, the remaining hESCs within the colonies continue to proliferate and remain pluripotent. Xu, et al., Nat Biotechnol 19: 971-974, 2001. This suggests that this fibroblastic hESC derivative can function in a similar fashion to the feeder layers that are routinely used to maintain hESC in an undifferentiated state. Compositions and methods are provided herein for the isolation, propagation and analysis of this subpopulation of cells. The subpopulation of cells expresses markers of extraembryonic endoderm, e.g., primitive endoderm, parietal endoderm, or visceral endoderm, including GATA-6 and characteristic ECM components. In addition, these cells, termed “extraembryonic endoderm-like” cells, and medium conditioned by extraembryonic endoderm-like cells, support the clonal growth of undifferentiated hESCs. Proteomic analysis (Mudpit) indicated that the extraembryonic endoderm-like cells secrete a distinct group of proteins, which include ECM proteins and a group of growth factors that includes inducers of the TGFβ/activin/nodal signaling pathway, which has been reported to support hESC self-renewal in vitro. Beattie, et al., Stem Cells 23: 489-495, 2005; James, Levine, Besser, & Hemmati-Brivanlou, Development 132: 1273-1282, 2005. These results support the idea that extraembryonic endoderm-like cells are an in vitro counterpart of the extraembryonic endoderm cells in the blastocyst, and suggest that they play a similar role in maintaining the pluripotence and proliferation of neighboring cells. A new hESC line has been derived using the extraembryonic endoderm-like cells to maintain undifferentiated colonies. The extraembryonic endoderm-like cells and conditioned medium from the extraembryonic endoderm-like cells are a source of material to identify the essential components required for maintenance of hESC pluripotence.

The present invention provides isolated mammalian non-immortalized extraembryonic endoderm-like cell line. The extraembryonic endoderm-like cell line comprises a gene and protein expression profile having decreased expression of genes associated with undifferentiated mammalian embryonic stem cells and increased expression of genes associated with extraembryonic endoderm. In one aspect, the gene and protein expression profile of the isolated extraembryonic endoderm-like cell line decreases expression of genes POU5F1/Oct4, LIN28, DNMT3B, ZIC2, ZIC3, and UTF1, and increases expression of genes GATA6, DAB-2, basement membrane genes, laminin (LAMC1), collagens, fibronectin (FN1), and nidogens, compared to mammalian embryonic stem cells. A cell conditioned medium is provided derived from growth of an isolated mammalian extraembryonic endoderm-like cell line.

An isolated cell population is provided which is obtained by differentiating primate pluripotent stem cells, in which at least 5% of the cells express a gene or protein expression profile having decreased expression of one or more of POU5F1/Oct4, LIN28, DNMT3B, ZIC2, ZIC3, and UTF1, and having increased expression one or more of GATA6, DAB-2, basement membrane genes, laminin (LAMC1), collagens, fibronectin (FN1), and nidogens. In one aspect of the isolated cell population, at least 5% of the cells express at least two of the following markers: GATA6, DAB-2, basement membrane genes, laminin (LAMC1), collagens, fibronectin (FN1), and nidogens. In a further aspect, the isolated cell population comprises less than 1% undifferentiated pluripotent stem cells. In a further aspect, the embryonic stem cells are human embryonic stem cells. The isolated cell population can be extraembryonic-endoderm-like cells. The isolated cell population can further be a primate extraembryonic-endoderm-like cell population. The isolated cell population can further be a human extraembryonic-endoderm-like cell population. In a further aspect, the isolated cell population is primitive endoderm, parietal endoderm, or visceral endoderm

An isolated mammalian extraembryonic endoderm-like cell line is provided as deposited with the American Type Culture Collection, 10801 University Boulevard, Manassas, Va. 20110-2209 under the Budapest Treaty on Dec. 19, 2006 and given the Accession No. indicated: ATCC Accession Number ______.

A set of at least two isolated cell populations is provided consisting of: a first cell population comprising one or more primate pluripotent stem cells isolated from a primate preimplantation primate embryo or cells thereof, and a second cell population that proliferates in culture, comprising at least 30% pluripotent stem cell-derived extraembryonic endoderm-like cells, identifiable by a criteria that the extraembryonic endoderm-like cells express one or more of the following: GATA6, DAB-2, basement membrane genes, laminin (LAMC1), collagens, fibronectin (FN1), or nidogens. In one aspect, the first cell population is isolated from the primate preimplantation primate embryo or cells thereof having a normal or non-disease state. In a further aspect, the first cell population is isolated from the primate preimplantation primate embryo having a disease state. The disease state can be a genetic disease. In a detailed aspect, the disease state is Down's syndrome, Huntington's disease, or Lesch-Nyhan disease.

The set of two isolated cell populations can further comprise at least 60% pluripotent stem cell-derived extraembryonic endoderm-like cells. The set of two isolated cell populations can further comprise at least 90% pluripotent stem cell-derived extraembryonic endoderm-like cells. In one aspect, the pluripotent stem cells are embryonic stem cells. In a further aspect, the pluripotent stem cells are human pluripotent stem cells. The pluripotent stem cells can be, for example, human embryonic stem cells. In one aspect, the first population is one primate pluripotent stem cell. In a further aspect, the one or more pluripotent stem cells are derived from inner cell mass cells. The extraembryonic endoderm-like cells can express two or more of the following: GATA6, DAB-2, basement membrane genes, laminin (LAMC1), collagens, fibronectin (FN1), or nidogens. In one aspect, the medium preconditioned by the extraembryonic endoderm-like cells causes proliferation of human embryonic stem cells without differentiation. In a further aspect, the second cell population has been obtained by culturing the pluripotent stem cells on an extracellular matrix on a solid substrate, and selecting cells having said criteria.

A culture system for maintaining undifferentiated growth of human embryonic stem cells is provided which comprises a substrate covered with human embryonic stem cell-derived extraembryonic endoderm-like feeder cells and one or more undifferentiated human embryonic stem cells. In one aspect, at least 60% of the human embryonic stem cells remain substantially undifferentiated after 20 passages. In a further aspect, at least 78% of the human embryonic stem cells remain substantially undifferentiated after 20 passages. The undifferentiated human embryonic stem cells can derive from one human embryonic stem cell. In a further aspect, the one or more undifferentiated human embryonic stem cells derive from inner cell mass cells.

A culture system for maintaining undifferentiated growth of human embryonic stem cells is provided which comprises a medium preconditioned by extraembryonic endoderm-like feeder cells, and one or more undifferentiated human embryonic stem cells. In one aspect, at least 60% of the human embryonic stem cells remain substantially undifferentiated after 20 passages. In a further aspect, at least 80% of the human embryonic stem cells remain substantially undifferentiated after 20 passages. The undifferentiated human embryonic stem cells can derive from one human embryonic stem cell. In a further aspect, the one or more undifferentiated human embryonic stem cells derive from inner cell mass cells.

A method for culturing undifferentiated mammalian cells is provided which comprises obtaining a single undifferentiated mammalian embryonic stem cell, and inoculating the single cell onto mammalian extraembryonic endoderm-like feeder cells in a nutrient medium. In one aspect, the mammalian embryonic stem cells are human, primate, mouse, or rat. In a further aspect, the mammalian extraembryonic endoderm-like feeder cells are human, primate, mouse, or rat. The human embryonic stem cells can be WA09 human embryonic stem cell line. The extraembryonic endoderm-like feeder cells can be positive for cell markers of one or more of the following: GATA6, DAB-2, basement membrane genes, laminin (LAMC1), collagens, fibronectin (FN1), or nidogens. In one aspect, the method is used for establishing a clonal human embryonic stem cell line. In a further aspect, the method is suitable for gene transfection.

The method for culturing undifferentiated mammalian cells can further comprise obtaining a single undifferentiated human embryonic stem cell which comprises selecting a group of undifferentiated cells from a cell culture, and dissociating the group of undifferentiated cells into single cells. In one aspect, the dissociation method is enzymatic degradation. In a detailed aspect, the enzymatic degradation is collagenase degradation.

A method for generating isolated primate extraembryonic endoderm-like cells is provided which comprises growing primate embryonic stem cells on extracellular matrix under feeder cell free conditions, identifying extraembryonic endoderm-like cells as positive for cell markers of one or more of the following: GATA6, DAB-2, basement membrane genes, laminin (LAMC1), collagens, fibronectin (FN1), or nidogens, and isolating extraembryonic endoderm-like cells from the embryonic stem cells. In one aspect, the primate embryonic stem cells are human embryonic stem cells. The method can further comprise isolating extraembryonic endoderm-like cells from the embryonic stem cells by mechanical dissection. The method can further comprise isolating extraembryonic endoderm-like cells from the embryonic stem cells by enzymatic digestion.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIGS. 1A, 1B, 1C, 1D, 1E, 1F, 1G, 1H, 1I, 1J, 1K, 1L, 1M, 1N and 1O show primitive endoderm-like cells from undifferentiated hESCs.

FIGS. 2A, 2B, 2C and 2D show characterization of PEL Cells

FIGS. 3A, 3B, 3C, 3D, 3E, 3F, 3G, 3H, and 31 show hESC-derived PEL cells support growth of single hESCs into clonal colonies.

FIG. 4 shows karyotypic analysis of hESC cultured on mitotically inactivated PEL cells.

FIGS. 5A, 5B, 5C, 5D, 5E, 5F, 5G, 5H and 5I show comparison of markers in hESCs and PEL cells by immunocytochemistry and gene expression microarray.

FIGS. 6A and 6B shows surface markers expressed on the surface of PEL cells.

FIG. 7 shows RT-PCR analysis of PEL, hESC, and HFF.

FIGS. 8A, 8B, 8C, 8D, 8E and 8F show immunocytochemical analysis of AFP in cultures of PEL cells differentiated into visceral endoderm.

### DETAILED DESCRIPTION

### Overview

The present invention provides compositions comprising an isolated cell population characterized as a mammalian extraembryonic endoderm-like cell population. Primate or human embryonic stem cells (ESCs) spontaneously generate the primate or human extraembryonic endoderm-like cells wherein the extraembryonic endoderm-like cells sustain the pluripotence of the primate or human ESCs. Methods for isolating an extraembryonic endoderm-like cell population are provided. Methods for culturing undifferentiated human embryonic stem cells are provided which comprise obtaining a single undifferentiated human embryonic stem cell, and inoculating the single cell onto extraembryonic endoderm-like feeder cells in a nutrient medium.

One of the first cell lineages to emerge during embryogenesis is the extraembryonic primitive endoderm (PE), which maintains the inner cell mass (ICM) and further differentiates into extraembryonic visceral endoderm. We observed that pluripotent ICM-derived cultured human embryonic stem cells (hESCs) spontaneously generate a clonally-related population of extraembryonic PE-like (PEL) cells that maintain pluripotence of hESC. Purified expanded subpopulations of PEL cells were characterized by immunocytochemistry, whole genome gene expression, protein profiling, and their ability to differentiate further into visceral endoderm. PEL cell-conditioned medium (CM) supports efficient clonal expansion of single pluripotent hESCs, and proteomic analysis of CM identified extrinsic factors that can maintain pluripotence, including ECM proteins, proteases and protease inhibitors, and regulators of IGF, WNT, and TGF-beta/activin/nodal signaling pathways. Signaling between PEL cells and hESCs in vitro can emulate the in vivo interaction between PE and the ICM and thus give insight into the earliest events in human development.

A subpopulation of hESC-derived early-differentiating cells were isolated, propagated, and analyzed. The cell population shows characteristics of extraembryonic endoderm, including expression of GATA-6 and other markers associated with the primitive endoderm. In addition, the present invention demonstrates that these cells, termed extraembryonic endoderm-like cells or “primitive endoderm-like” (PEL) cells, and medium conditioned by PEL cells, support the clonal growth of undifferentiated hESCs. Large scale proteomic analysis indicates that the PEL cells secrete a distinct group of proteins, which include ECM proteins and a group of growth factor-associated proteins that includes Inhibin beta A/Activin A, an inducer of the TGFβ/activin/nodal signaling pathway, which has been reported to support hESC self-renewal in vitro. Beattie, G. M. et al., Stem Cells 23, 489-495, 2005; James, D., et al., Development 132, 1273-1282, 2005. The results imply that PEL cells (which are clonally-related to the hESCs) are acting as in vitro counterpart of the early differentiating extraembryonic endoderm cells that maintain the ICM in the blastocyst, and suggest that they play a similar role in maintaining the pluripotence of hESCs in culture. Furthermore, the characterization of PEL cells raises the intriguing possibility that the diverse fibroblast types that are used as feeder layers for hESC culture can owe their supportive function to their resemblance to the embryonic primitive endoderm.

It is to be understood that this invention is not limited to particular methods, reagents, compounds, compositions or biological systems, which can, of course, vary. It is also to be understood that the terminology used herein is for the purpose of describing particular aspects only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Pluripotent stem cells” (PS cells) or “pluripotent embryonic stem cells” (ESCs) from primates or humans are cells derived from any kind of embryonic tissue (fetal or pre-fetal tissue). “Pluripotent stem cells” or “pluripotent embryonic stem cells” (ESCs) have the ability to self replicate for indefinite periods have the characteristic of being capable under appropriate conditions of producing progeny of different cell types that are derivatives of all of the 3 germinal layers (endoderm, mesoderm, and ectoderm), according to a standard art-accepted test, such as the ability to form a teratoma in 8-12 week old SCID mice, or the ability to form identifiable cells of all three germ layers in tissue culture.

Included in the definition of PS cells or ESCs are embryonic cells of various types, exemplified by human embryonic stem (hESC) cells, described by Thomson, et al., Science 282:1145, 1998; embryonic stem cells from other primates, such as Rhesus stem cells (Thomson, et al., Proc. Natl. Acad. Sci. USA 92: 7844, 1995), marmoset stem cells (Thomson et al., Biol. Reprod. 55: 254, 1996) and human embryonic germ (hEG) cells (Shamblott, et al., Proc. Natl. Acad. Sci. USA 95: 13726, 1998). Other types of pluripotent cells, e.g., mammalian pluripotent stem cells, are also included in the term. Any cells of mammalian or primate origin that are capable of producing progeny that are derivatives of all three germinal layers are included, regardless of whether they were derived from embryonic tissue, fetal tissue, or other sources. The PS cells or ESCs are not derived from a malignant source. It is desirable (but not always necessary) that the cells be karyotypically normal.

An “extraembryonic endoderm-like cell” refers to cells that have down-regulated pluripotence-associated genes and begin to express genes associated with the extraembryonic endoderm cell lineage, for example, primitive endoderm lineage (e.g., a primitive endoderm-like [PEL] cell line), parietal endoderm lineage, or visceral endoderm lineage, including, but not limited to, genes for extracellular matrix (ECM) components and the transcription factor, GATA-6. Further characteristics of extraembryonic endoderm-like cells are described herein.

A “variant” as used herein refers to any “variant” of a specified cell line (i.e., variant cell line) including progeny of the specified cell line, a modified or mutated cell line obtained or derived from the specified cell line or its progeny, or other recipient cell line that contains genetic material obtained directly or indirectly from the specified cell line. Such a variant cell line can, for example, be formed by removing genetic material from a specified microorganism or cell line and subsequently introducing it into a cell line (i.e., the progeny or other recipient cell line) by any conventional methodology including, but not limited to, dextran-mediated transfection, calcium phosphate precipitation, polybrene mediated transfection, protoplast fusion, electroporation, encapsulation of the polynucleotide(s) in liposomes, direct microinjection of the DNA into nuclei, transduction, differentiation and the like. A variant can be formed by introducing one or more mutations or modifications into the genome or other genetic material (e.g., vectors, plasmids, extrachromosomal elements, and the like) of a cell line. Such mutations or modifications can include one or more insertion mutations, deletion mutations and/or substitutions or various combinations thereof. The mutations or modifications can be insertions into the genome or other genetic material (e.g., vectors, plasmids, extrachromosomal elements, and the like) of the cell line. Alternatively, the mutations can be deletions of one or more bases and/or nucleic acid sequences from the genome or other genetic material (e.g., vectors, plasmids, extrachromosomal elements, and the like) of the cell line. In some instances, the mutations can be the alteration of one or more bases in the genome of the cell line. Such modifications or mutations can also comprise, for example, methylating or possibly substituting one or more nucleic acid bases and/or nucleic acid molecules for other nucleic acid molecules and/or bases. In addition, one cell line is a variant of a parent cell line if it contains the genome of the parent cell line but does not contain some or all of the same extrachromosomal nucleic acid molecules. Variants of a cell line of the invention can also include those cell lines obtained by the addition of one or more nucleic acid molecules into the cell line of interest. Nucleic acid molecules which can be introduced into a cell line will be recognized by one skilled in the art and can include, but are not limited to, vectors, plasmids, oligonucleotides, RNA, DNA, RNA/DNA hybrids, phage sequences, virus sequences, regardless of the form or conformation (e.g., linear, circular, supercoiled, single stranded, double stranded, single/double stranded hybrids and the like). Examples of mutations or other genetic alterations which can be incorporated into the cell line of the present invention include, but are not limited to, mutations or alterations that create: a cell line resistant to antibiotic selection, a cell line with increased permissiveness to transfection; a cell line with increased expression of transgenes; genomic incorporation of a gene of interest in a cell line; and genomic incorporation and amplification of a gene of interest in a cell line. Other suitable modifications are known to those skilled in the art and such modifications are considered to be within the scope of the present invention.

“Non-immortalized” refers to a cell line that has not been genetically altered, for example, by gene transfection, to form an immortalized cell line.

PS cell cultures are described as “undifferentiated” when a substantial proportion of stem cells and their derivatives in the population display morphological characteristics of undifferentiated cells, clearly distinguishing them from differentiated cells of embryo or adult origin. Undifferentiated PS or ESC cells are easily recognized by those skilled in the art, and typically appear in the two dimensions of a microscopic view in colonies of cells with high nuclear/cytoplasmic ratios and prominent nucleoli. It is understood that colonies of undifferentiated cells within the population will often be surrounded by neighboring cells that are differentiated.

In the context of cell ontogeny, the term “differentiated” is a relative term. A “differentiated cell” is a cell that has progressed further down the developmental pathway than the cell it is being compared with. Thus, pluripotent embryonic stem cells can differentiate to lineage-restricted precursor cells (such as a extraembryonic endoderm-like cell or a mesenchymal stem cell), which in turn can differentiate into other types of precursor cells further down the pathway (such as an osteoblast precursor), and then to an end-stage differentiated cell, which plays a characteristic role in a certain tissue type, and can or can not retain the capacity to proliferate further.

“Feeder cells” or “feeders” refers to cells of one tissue type that are co-cultured with cells of another tissue type, to provide an environment in which cells of the second tissue type can grow. The feeder cells are optionally from a different species as the cells they are supporting. For example, primary cultures of extraembryonic endoderm-like cells of the present invention provide feeder cells that can support the culture of primate or human PS cells, or primate or human embryonic stem cells (ESCs). PS or ESC cell populations are said to be “essentially free” of feeder cells if the cells have been grown through at least one round after splitting in which fresh feeder cells are not added to support the growth of the pPS.

A “growth environment” is an environment in which cells of interest will proliferate, differentiate, or mature in vitro. Features of the environment include the medium in which the cells are cultured, any growth factors or differentiation-inducing factors that can be present, and a supporting structure (such as a substrate on a solid surface) if present.

“ECM” refers to a cellular matrix composed of extracellular and cellular matrices isolated from feeder cells. Extraembryonic endoderm-like cells of the present invention are provided as feeder cells that secrete an extracellular matrix and other growth factors that support the self renewal of PS cells or pluripotent human embryonic stem cells (hESCs).

“Conditioned medium” refers to a medium harvested after the extraembryonic endoderm-like cells or feeder cells have been cultivated within for a period of time. The conditioned medium of the present invention can then be used to cultivate hESCs, for it contains many mediator substances, such as growth factors and cytokines, that were secreted by the feeder cells cultivated previously and can thus help promote the growth of hESCs. In one aspect, the conditioned medium is a medium harvested after the feeder cells have been cultivated in it for at least 1 day. Isolated growth factors from the cell conditioned medium of mammalian, primate, or human extraembryonic endoderm-like cells comprise one or more factors from the following: ECM proteins, laminin 1, collagen IV isotypes, proteases, protease inhibitors, cell surface adhesion proteins, cell-signaling proteins, cadherins, chloride intracellular channel 1, transmembrane receptor PTK7, growth factors, insulin-like growth factor, Inhibin beta A, inducers of the TGFβ/Activin/nodal signaling pathway, and Activin A.

“Anchorage-dependent cells” refers to cells or mammalian cells that have a requirement for replication in tissue culture that the cells attach to a surface, e.g., a tissue culture flask surface.

“Essentially”, “essentially effective”, or “essentially pure” refers to a population of cells or a method which is at least 20+%, 30+%, 40+%, 50+%, 60+%, 70+%, 80+%, 85+%, 90+%, or 95+% effective, more preferably at least 98+% effective, most preferably 99+% effective. Therefore, a method that enriches for a given cell population, enriches at least about 20+%, 30+%, 40+%, 50+%, 60+%, 70+%, 80%, 85%, 90%, or 95% of the targeted cell population, most preferably at least about 98% of the cell population, most preferably about 99% of the cell population. In certain aspects the cells in an enriched population of extraembryonic endoderm-like cells of the invention comprise a population of cells which have a distinct proteomtic profile, for example, expression of ECM proteins and GATA-6 protein.

“Isolated” refers to a cell, cellular component, or a molecule that has been removed from its native environment. “Isolated” or “purified” refers to altered “by the hand of man” from the natural state i.e., anything that occurs in nature is defined as isolated when it has been removed from its original environment, or both. “Isolated” also defines a composition, for example, a extraembryonic endoderm-like cell population, that is separated from contaminants (i.e. substances that differ from the cell). In an aspect, a population or composition of cells is substantially free of cells and materials with which it can be associated in nature. “Isolated” or “purified” or “substantially pure”, with respect to extraembryonic endoderm-like cells, refers to a population of extraembryonic endoderm-like cells that is at least about 50%, at least about 75%, preferably at least about 85%, more preferably at least about 90%, and most preferably at least about 95% pure, with respect to extraembryonic endoderm-like cells making up a total cell population. Recast, the term “substantially pure” refers to a population of extraembryonic endoderm-like cells of the present invention that contain fewer than about 50%, preferably fewer than about 30%, preferably fewer than about 20%, more preferably fewer than about 10%, most preferably fewer than about 5%, embryonic stem cells (primate or human) or lineage-committed cells other than extraembryonic endoderm-like cells in the original unamplified and isolated population prior to subsequent culturing and amplification. Purity of a population or composition of cells can be assessed by appropriate methods that are well known in the art.

“Gene therapy” refers to the transfer and stable insertion of new genetic information into cells, e.g., PS cells, ESCs, or extraembryonic endoderm-like cells, for the therapeutic treatment of diseases or disorders. A variety of means for administering gene therapy to a mammalian subject will, in view of this specification, be apparent to those of skill in the art. Gene therapy techniques are described in the specification. A foreign gene is transferred into a cell that proliferates to introduce the transferred gene throughout the cell population. Therefore, cells and compositions of the invention can be the target of gene transfer, since they will produce various lineages which will potentially express the foreign gene. A cell is said to be “genetically altered” when a polynucleotide has been transferred into the cell by any suitable means of artificial manipulation, or where the cell is a progeny of the originally altered cell that has inherited the polynucleotide. The polynucleotide will often comprise a transcribable sequence encoding a protein of interest, which enables the cell to express the protein at an elevated level. The genetic alteration is said to be “inheritable” if progeny of the altered cell have the same alteration.

“Antibody” as used in this disclosure refers to both polyclonal and monoclonal antibody. The ambit of the term deliberately encompasses not only intact immunoglobulin molecules, but also such fragments and derivatives of immunoglobulin molecules (such as single chain Fv constructs, diabodies, and fusion constructs) as can be prepared by techniques known in the art, and retaining a desired antibody binding specificity.

### Oligonucleotide Arrays

A substrate comprising a plurality of oligonucleotide primers or probes of the invention can be used, e.g., to detect expression of a plurality of any of the herein-provided mRNAs to characterize a cell population comprising extraembryonic endoderm-like cells, e.g., primitive endoderm-like cells, or to detect the expression of one or more of the present mRNAs to characterize a cell population comprising extraembryonic endoderm-like cells, optionally in conjunction with the expression of one or more heterologous genes.

Any polynucleotide provided herein can be attached in overlapping areas or at random locations on the solid support. Alternatively the polynucleotides of the invention useful to characterize a cell population comprising extraembryonic endoderm-like cells, can be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular aspect of these polynucleotide arrays is known as the Genechips™, and has been generally described in U.S. Pat. No. 5,143,854; PCT publications WO 90/15070 and 92/10092. These arrays can generally be produced using mechanical synthesis methods or light directed synthesis methods which incorporate a combination of photolithographic methods and solid phase oligonucleotide synthesis. Fodor et al., Science 251: 767-73, 1991. The immobilization of arrays of oligonucleotides on solid supports has been rendered possible by the development of a technology generally identified as “Very Large Scale Immobilized Polymer Synthesis” (VLSIPS™; Fodor et al., 1991) in which, typically, probes are immobilized in a high density array on a solid surface of a chip. Examples of VLSIPS™ technologies are provided in U.S. Pat. Nos. 5,143,854; and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

Consequently, the invention concerns an array of nucleic acid molecules comprising at least one polynucleotide described above as probes and primers. Preferably, the invention concerns an array of nucleic acid comprising at least two polynucleotides described above as probes and primers.

### Synthesis of Probe Arrays

Arrays of probes to detect expression patterns in extraembryonic endoderm-like cells, PS cells, or ESCs, can be synthesized in a step-by-step manner on a support or can be attached in presynthesized form. A preferred method of synthesis is VLSIPS™ (see Fodor et al., Nature 364: 555-556, 1993; McGall et al., U.S. Ser. No. 08/445,332; U.S. Pat. No. 5,143,854; EP 476,014), which entails the use of light to direct the synthesis of polynucleotide probes in high-density, miniaturized arrays. Algorithms for design of masks to reduce the number of synthesis cycles are described by Hubbel et al., U.S. Pat. No. 5,571,639 and U.S. Pat. No. 5,593,839. Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id.; Pease et al., EP 728,520.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stem et al., U.S. Pat. No. 5,631,734 and are available from Affymetrix, Inc., under the GeneChip™ label. Some types of label provide a signal that can be amplified by enzymatic methods (see Broude et al, Proc. Natl. Acad. Sci. USA. 91: 3072-3076, 1994).

### Design of Arrays

Customized and Generic Arrays. The design of arrays for expression monitoring, e.g., in extraembryonic endoderm-like cells, PS cells, or ESCs, is generally described, for example, WO 97/27317 and WO 97/10365 (these references are herein incorporated by reference). There are two principal categories of arrays. One type of array detects the presence and/or levels of particular mRNA sequences that are known in advance. In these arrays, polynucleotide probes can be selected to hybridize to particular preselected subsequences of mRNA gene sequence. Such expression monitoring arrays can include a plurality of probes for each mRNA to be detected. For analysis of mRNA nucleic acids, the probes are designed to be complementary to the region of the mRNA that is incorporated into the nucleic acids (i.e., the 3′ end). The array can also include one or more control probes.

Generic arrays can include all possible nucleotides of a given length; that is, polynucleotides having sequences corresponding to every permutation of a sequence. Thus since the polynucleotide probes of this invention preferably include up to 4 bases (A, G, C, T) or (A, G, C, U) or derivatives of these bases, an array having all possible nucleotides of length X contains substantially 4.sup.X different nucleic acids (e.g., 16 different nucleic acids for a 2 mer, 64 different nucleic acids for a 3 mer, 65536 different nucleic acids for an 8 mer). Some small number of sequences can be absent from a pool of all possible nucleotides of a particular length due to synthesis problems, and inadvertent cleavage). An array comprising all possible nucleotides of length X refers to an array having substantially all possible nucleotides of length X. All possible nucleotides of length X includes more than 90%, typically more than 95%, preferably more than 98%, more preferably more than 99%, and most preferably more than 99.9% of the possible number of different nucleotides. Generic arrays are particularly useful for comparative hybridization analysis between two mRNA populations or nucleic acids derived therefrom.

Variations. Either customized or generic probe arrays can contain control probes in addition to the probes described above.

Normalization Controls. Normalization controls are typically perfectly complementary to one or more labeled reference polynucleotides that are added to the nucleic acid sample. The signals obtained from the normalization controls after hybridization provide a control for variations in hybridization conditions, label intensity, reading and analyzing efficiency and other factors that can cause the signal of a perfect hybridization to vary between arrays. Signals (e.g., fluorescence intensity) read from all other probes in the array can be divided by the signal (erg., fluorescence intensity) from the control probes thereby normalizing the measurements.

Virtually any probe can serve as a normalization control. However, hybridization efficiency can vary with base composition and probe length. Normalization probes can be selected to reflect the average length of the other probes present in the array, however, they can also be selected to cover a range of lengths. The normalization control(s) can also be selected to reflect the (average) base composition of the other probes in the array. However one or a fewer normalization probes can be used and they can be selected such that they hybridize well (i.e., no secondary structure) and do not match any target-specific probes.

Normalization probes can be localized at any position in the array or at multiple positions throughout the array to control for spatial variation in hybridization efficiently. The normalization controls can be located at the corners or edges of the array as well as in the middle of the array.

Expression Level Controls. Expression level controls can be probes that hybridize specifically with constitutively expressed genes in the biological sample. Expression level controls can be designed to control for the overall health and metabolic activity of a cell. Examination of the covariance of an expression level control with the expression level of the target nucleic acid can indicate whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse can also be true. Thus where the expression levels of both an expression level control and the target gene appear to both decrease or to both increase, the change can be attributed to changes in the metabolic activity of the cell as a whole, not to differential expression of the target gene in question. Conversely, where the expression levels of the target gene and the expression level control do not covary, the variation in the expression level of the target gene can be attributed to differences in regulation of that gene and not to overall variations in the metabolic activity of the cell.

Virtually any constitutively expressed gene can provide a suitable target for expression level controls. Typically expression level control probes can have sequences complementary to subsequences of constitutively expressed genes including, but not limited to the B-actin gene, the transferrin receptor gene, the GAPDH gene, and the like.

Mismatch Controls. Mismatch controls can also be provided for the probes to the target genes, for expression level controls or for normalization controls. Mismatch controls are typically employed in customized arrays containing probes matched to known mRNA species. For example, some such arrays contain a mismatch probe corresponding to each match probe. The mismatch probe is the same as its corresponding match probe except for at least one position of mismatch. A mismatched base is a base selected so that it is not complementary to the corresponding base in the target sequence to which the probe can otherwise specifically hybridize. One or more mismatches are selected such that under appropriate hybridization conditions (e.g. stringent conditions) the test or control probe can be expected to hybridize with its target sequence, but the mismatch probe cannot hybridize (or can hybridize to a significantly lesser extent). Mismatch probes can contain a central mismatch. Thus, for example, where a probe is a 20 mer, a corresponding mismatch probe can have the identical sequence except for a single base mismatch (e.g., substituting a G, a C or a T for an A) at any of positions 6 through 14 (the central mismatch).

In generic (e.g., random, arbitrary, or haphazard) arrays, since the target nucleic acid(s) are unknown perfect match and mismatch probes cannot be a priori determined, designed, or selected. In this instance, the probes can be provided as pairs where each pair of probes differ in one or more preselected nucleotides. Thus, while it is not known a priori which of the probes in the pair is the perfect match, it is known that when one probe specifically hybridizes to a particular target sequence, the other probe of the pair can act as a mismatch control for that target sequence. The perfect match and mismatch probes need not be provided as pairs, but can be provided as larger collections (e.g., 3, 4, 5, or more) of probes that differ from each other in particular preselected nucleotides.

In both customized and generic arrays mismatch probes can provide a control for non-specific binding or cross-hybridization to a nucleic acid in the sample other than the target to which the probe is complementary. Mismatch probes thus can indicate whether a hybridization is specific or not. For example, if the complementary target is present the perfect match probes can be consistently brighter than the mismatch probes. In addition, if all central mismatches are present, the mismatch probes can be used to detect a mutation. Finally, the difference in intensity between the perfect match and the mismatch probe (I(PM)-I(MM)) can provide a good measure of the concentration of the hybridized material.

Sample Preparation, Amplification, and Quantitation Controls. Arrays can also include sample preparation/amplification control probes. These can be probes that are complementary to subsequences of control genes selected because they do not normally occur in the nucleic acids of the particular biological sample being assayed. Suitable sample preparation/amplification control probes can include, for example, probes to bacterial genes (e.g., Bio B) where the sample in question is a biological sample from a eukaryote.

The RNA sample can then be spiked with a known amount of the nucleic acid to which the sample preparation/amplification control probe is directed before processing. Quantification of the hybridization of the sample preparation/amplification control probe can then provide a measure of alteration in the abundance of the nucleic acids caused by processing steps (e.g., PCR, reverse transcription, or in vitro transcription).

Quantitation controls can be similar. Typically they can be combined with the sample nucleic acid(s) in known amounts prior to hybridization. They are useful to provide a quantitation reference and permit determination of a standard curve for quantifying hybridization amounts (concentrations).

### Methods of Detection

In one method of detection, extraembryonic endoderm-like cell mRNA, PS cell mRNA, or ESC mRNA or nucleic acid derived therefrom, typically in denatured form, are applied to an array. The component strands of the nucleic acids hybridize to complementary probes, which are identified by detecting label. Optionally, the hybridization signal of matched probes can be compared with that of corresponding mismatched or other control probes. Binding of mismatched probe serves as a measure of background and can be subtracted from binding of matched probes. A significant difference in binding between a perfectly matched probes and a mismatched probes signifies that the nucleic acid to which the matched probes are complementary is present. Binding to the perfectly matched probes is typically at least 1.2, 1.5, 2, 5 or 10 or 20 times higher than binding to the mismatched probes.

In a variation of the above method, nucleic acids are not labeled but are detected by template-directed extension of a probe hybridized to a nucleic acid strand with the nucleic acid strand serving as a template. The probe is extended with a labeled nucleotide, and the position of the label indicates, which probes in the array have been extended. By performing multiple rounds of extension using different bases bearing different labels, it is possible to determine the identity of additional bases in the tag than are determined through complementarity with the probe to which the tag is hybridized. The use of target-dependent extension of probes is described by U.S. Pat. No. 5,547,839.

In a further variation, probes can be extended with inosine. The inosine strand can be labeled. The addition of degenerate bases, such as inosine (it can pair with all other bases), can increase duplex stability between the polynucleotide probe and the denatured single stranded DNA nucleic acids. The addition of 1-6 inosines onto the end of the probes can increase the signal intensity in both hybridization and ligation reactions on a generic ligation array. This can allow for ligations at higher temperatures. The use of degenerate bases is described in WO 97/27317.

Ligation reactions can offer improved discriminate between fully complementary hybrids and those that differ by one or more base pairs, particularly in cases where the mismatch is near the 5′ terminus of the polynucleotide probes. Use of a ligation reaction in signal detection increases the stability of the hybrid duplex, improves hybridization specificity (particularly for shorter polynucleotide probes (e.g., 5 to 12-mers), and optionally, provides additional sequence information. Ligation reactions used in signal detection are described in WO 97/27317. Optionally, ligation reactions can be used in conjunction with template-directed extension of probes, either by inosine or other bases.

### Analysis of Hybridization Patterns

Proteomic analysis of a complex mixture utilizes MudPIT (multidimensional protein identification technology). MudPIT is a non-gel approach for the identification of proteins from complex mixtures. The technique consists of a 2-dimensional chromatography separation, prior to electrospray mass spectrometry. By exploiting a peptide's unique physical properties of charge and hydrophobicity, complex mixtures can be separated prior to sequencing by tandem mass spectroscopy. The first dimension is normally a strong cation exchange (SCX) column, as these have high loading capacities. The second dimension is reverse phase chromatography (RP), which complements the SCX as it is efficient at removing salts and has the added advantage of being compatible with electrospray mass spectrometry.

Sample preparation requires that the samples are denatured, the cysteines reduced and alkylated and the proteins digested with a protease such as trypsin. The samples are then acidified and loaded onto the SCX column. Charged peptides bind to the SCX column, whereas any uncharged peptides pass through and bind to a reverse phase trap column. The peptides are then eluted from the trap column onto an analytical RP column, using a reverse phase gradient, separated and eluted into a tandem mass spectrometer. Peptide fragmentation data is then obtained to identify the peptides and hence the proteins from which they are derived. In the next step, salt at a particular concentration is injected onto the SCX column, displacing further peptides from it onto the RP trap column. Salt is removed by washing and again an analytical RP separation is performed and the eluting peptides analysed by mass spectrometry. Incremental increases of salt are used (salt step gradient from around 0-200 mM). The end result is multiple protein identifications from each salt step. Expression analysis using Affymetrix microarrays was first described in a study of gene expression after induction of cytokine in mouse lymphocyte. Lockhart D. J., et al., Nat. Biotechnol., 14: 1675-1680, 1996; Golub, T. R., et al., Science., 286: 531-537, 1999; Winzeler, E. A. et al., Science., 285: 901-906, 1999.

The position of label is detected for each probe in the array using a reader, such as described by U.S. Pat. No. 5,143,854, WO 90/15070, and Trulson et al., supra. For customized arrays, the hybridization pattern can then be analyzed to determine the presence and/or relative amounts or absolute amounts of known mRNA species in samples being analyzed as described in e.g., WO 97/10365. Comparison of the expression patterns of two samples is useful for identifying mRNAs and their corresponding genes that are differentially expressed between the two samples.

The quantitative monitoring of expression levels for large numbers of genes can prove valuable in elucidating gene function, exploring the causes and mechanisms of disease, and for the discovery of potential therapeutic and diagnostic targets. Expression monitoring can be used to monitor the expression (transcription) levels of nucleic acids whose expression is altered in a disease state. For example, late-onset Alzheimer disease can be characterized by the underexpression or overexpression of a particular marker, for example, the underexpression of the gene encoding low density lipoprotein receptor-related protein 6 in the case of determining a prognosis or diagnosis of a human subject with late-onset Alzheimer disease.

Expression monitoring can be used to monitor expression of various genes in response to defined stimuli, such as a drug. This is especially useful in drug research if the end point description is a complex one, not simply asking if one particular gene is overexpressed or underexpressed. Therefore, where a disease state or the mode of action of a drug is not well characterized, the expression monitoring can allow rapid determination of the particularly relevant genes.

In generic arrays, the hybridization pattern is also a measure of the presence and abundance of relative mRNAs in a sample, although it is not immediately known, which probes correspond to which mRNAs in the sample.

However the lack of knowledge regarding the particular genes does not prevent identification of useful therapeutics. For example, if the hybridization pattern on a particular generic array for a healthy cell is known and significantly different from the pattern for a diseased cell, then libraries of compounds can be screened for those that cause the pattern for a diseased cell to become like that for the healthy cell. This provides a detailed measure of the cellular response to a drug.

Generic arrays can also provide a powerful tool for gene discovery and for elucidating mechanisms underlying complex cellular responses to various stimuli. For example, generic arrays can be used for expression fingerprinting. Suppose it is found that the mRNA from a certain cell type displays a distinct overall hybridization pattern that is different under different conditions (e.g., when harboring mutations in particular genes, in a disease state). Then this pattern of expression (an expression fingerprint), if reproducible and clearly differentiable in the different cases can be used as a very detailed diagnostic. It is not required that the pattern be fully interpretable, but just that it is specific for a particular cell state (and preferably of diagnostic and/or prognostic relevance).

Both customized and generic arrays can be used in drug safety studies. For example, if one is making a new antibiotic, then it should not significantly affect the expression profile for mammalian cells. The hybridization pattern can be used as a detailed measure of the effect of a drug on cells, for example, as a toxicological screen.

The sequence information provided by the hybridization pattern of a generic array can be used to identify genes encoding mRNAs hybridized to an array. Such methods can be performed using DNA nucleic acids of the invention as the target nucleic acids described in WO 97/27317. DNA nucleic acids can be denatured and then hybridized to the complementary regions of the probes, using standard conditions described in WO 97/27317. The hybridization pattern indicates which probes are complementary to nucleic acid strands in the sample. Comparison of the hybridization pattern of two samples indicates which probes hybridize to nucleic acid strands that derive from mRNAs that are differentially expressed between the two samples. These probes are of particular interest, because they contain complementary sequence to mRNA species subject to differential expression. The sequence of such probes is known and can be compared with sequences in databases to determine the identity of the full-length mRNAs subject to differential expression provided that such mRNAs have previously been sequenced. Alternatively, the sequences of probes can be used to design hybridization probes or primers for cloning the differentially expressed mRNAs. The differentially expressed mRNAs are typically cloned from the sample in which the mRNA of interest was expressed at the highest level. In some methods, database comparisons or cloning is facilitated by provision of additional sequence information beyond that inferable from probe sequence by template dependent extension as described above.

### Synthesis of Probe Arrays

Arrays of probes can be synthesized in a step-by-step manner on a support or can be attached in presynthesized form. A preferred method of synthesis is VLSIPS™ (see Fodor et al., Nature 364: 555-556, 1993; McGall et al., U.S. Ser. No. 08/445,332; U.S. Pat. No. 5,143,854; EP 476,014), which entails the use of light to direct the synthesis of polynucleotide probes in high-density, miniaturized arrays. Algorithms for design of masks to reduce the number of synthesis cycles are described by Hubbel et al., U.S. Pat. No. 5,571,639 and U.S. Pat. No. 5,593,839. Arrays can also be synthesized in a combinatorial fashion by delivering monomers to cells of a support by mechanically constrained flowpaths. See Winkler et al., EP 624,059. Arrays can also be synthesized by spotting monomers reagents on to a support using an ink jet printer. See id.; Pease et al., EP 728,520.

After hybridization of control and target samples to an array containing one or more probe sets as described above and optional washing to remove unbound and nonspecifically bound probe, the hybridization intensity for the respective samples is determined for each probe in the array. For fluorescent labels, hybridization intensity can be determined by, for example, a scanning confocal microscope in photon counting mode. Appropriate scanning devices are described by e.g., Trulson et al., U.S. Pat. No. 5,578,832; Stem et al., U.S. Pat. No. 5,631,734 and are available from Affymetrix, Inc., under the GeneChip™ label. Some types of label provide a signal that can be amplified by enzymatic methods (see Broude et al., Proc. Natl. Acad. Sci. U.S.A. 91: 3072-3076, 1994).

### Genetic Engineering of Extraembryonic Endoderm-Like Cells or Embryonic Stem Cells (ESCs)

The primate or human extraembryonic endoderm-like cells or ESCs of the invention can be engineered using any of a variety of vectors including, but not limited to, integrating viral vectors, e.g., retrovirus vector or adeno-associated viral vectors; non-integrating replicating vectors, e.g., papilloma virus vectors, SV40 vectors, adenoviral vectors; or replication-defective viral vectors. Other methods of introducing DNA into cells include the use of liposomes, electroporation, a particle gun, or by direct DNA injection.

Hosts cells are preferably transformed or transfected with DNA controlled by or in operative association with, one or more appropriate expression control elements such as promoter or enhancer sequences, transcription terminators, polyadenylation sites, among others, and a selectable marker.

Following the introduction of the foreign DNA, engineered cells can be allowed to grow in enriched media and then switched to selective media. The selectable marker in the foreign DNA confers resistance to the selection and allows cells to stably integrate the foreign DNA as, for example, on a plasmid, into their chromosomes and grow to form foci which, in turn, can be cloned and expanded into cell lines. This method can be advantageously used to engineer cell lines which express the gene product.

Any promoter can be used to drive the expression of the inserted gene. For example, viral promoters include, but are not limited to, the CMV promoter/enhancer, SV 40, papillomavirus, Epstein-Barr virus or elastin gene promoter. Preferably, the control elements used to control expression of the gene of interest should allow for the regulated expression of the gene so that the product is synthesized only when needed in vivo. If transient expression is desired, constitutive promoters are preferably used in a non-integrating and/or replication-defective vector. Alternatively, inducible promoters could be used to drive the expression of the inserted gene when necessary.

Inducible promoters include, but are not limited to, those associated with metallothionein and heat shock proteins. Examples of transcriptional control regions that exhibit tissue specificity have been described. For example, tissue specific promoters can be used.

The cells of the invention can be genetically engineered to “knock out” expression of factors that promote inflammation or rejection at the implant site. Negative modulatory techniques for the reduction of target gene expression levels or target gene product activity levels are discussed below. “Negative modulation,” as used herein, refers to a reduction in the level and/or activity of target gene product relative to the level and/or activity of the target gene product in the absence of the modulatory treatment. The expression of a gene native to a chondrocyte can be reduced or knocked out using a number of techniques including, for example, inhibition of expression by inactivating the gene completely (commonly termed “knockout”) using the homologous recombination technique. Usually, an exon encoding an important region of the protein (or an exon 51 to that region) is interrupted by a positive selectable marker, e.g., neo, preventing the production of normal mRNA from the target gene and resulting in inactivation of the gene. A gene can also be inactivated by creating a deletion in part of a gene, or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084).

Antisense, DNAzymes and ribozyme molecules which inhibit expression of the target gene can also be used in accordance with the invention to reduce the level of target gene activity. For example, antisense RNA molecules which inhibit the expression of major histocompatibility gene complexes (HLA) have been shown to be most versatile with respect to immune responses. Still further, triple helix molecules can be utilized in reducing the level of target gene activity.

These techniques are described in detail by L. G. Davis et al. (eds), 1994, Basic Methods in Molecular Biology, 2nd ed., Appleton & Lange, Norwalk, Conn., which is incorporated herein by reference.

Using any of the foregoing techniques, the expression of IL-1 can be knocked out in the cells of the invention to reduce the production of inflammatory mediators by the cells of the invention. Likewise, the expression of MHC class II molecules can be knocked out in order to reduce the risk of rejection of the implanted tissue.

Once the mammalian, primate, or human extraembryonic endoderm-like cells or ESCs of the invention have been genetically engineered, they can be directly implanted into the patient to allow for the amelioration of the symptoms of disease by producing an anti-inflammatory gene product such as, for example, peptides or polypeptides corresponding to the idiotype of neutralizing antibodies for GM-CSF, TNF, IL-1, IL-2, or other inflammatory cytokines.

Alternatively, the genetically engineered cells can be used to produce new tissue in vitro, which is then implanted in the subject, as described supra.

### Use of Extraembryonic Endoderm-Like Cells or Embryonic Stem Cells (ESCs) for Transplantation

The treatment methods of the subject invention involves the implantation of mammalian, primate, or human extraembryonic endoderm-like cells or ESCs, into individuals in need thereof. The cells of the present invention, for example, extraembryonic endoderm-like cells expressing ECM proteins and GATA-6 protein, and hESCs can be allogeneic or autologous and can be delivered to the site of therapeutic need or “home” to the site. The cells of the present invention can differentiate in situ or provide trophic support to endogenous cells. The appropriate cell implantation dosage in humans can be determined from existing information relating to either the activity of the cells for example erythropoietin production, or the density of cells to treat hematopoietic disease. From in vitro culture and in vivo animal experiments, the amount of hormones produced can be quantitated, and this information is also useful in calculating an appropriate dosage of implanted material. Additionally, the patient can be monitored to determine if additional implantation can be made or implanted material reduced accordingly.

To enhance the differentiation, survival or activity of implanted cells additional factors can be added including growth factors such as morphogenetic proteins or corticosteroids, antioxidants or anti-inflammatory agents such as cyclosporin, statins, rapamycin, p38 kinase inhibitors.

To enhance vascularization and survival of the transplanted cells angiogenic factors such as VEGF, PDGF or bFGF can be added either alone or in combination with endothelial cells or their precursors including CD34+, CD34+/CD117+ cells.

Primate or human extraembryonic endoderm-like cells or ESCs can be used to treat diseases or chronic conditions resulting in morbidity or reduced life expectancy. These conditions and diseases include, for example, cancer or neoplastic disease of hematopoietic tissue, autoimmune disease, or genetic diseases. Clinical management strategies, for example, frequently focus on the prevention of further damage or injury rather than replacement or repair of the damaged tissue (e.g., hematopoietic tissue, renal tubules, glomeruli, neurons, glial cells, cardiac muscle); include treatment with exogenous steroids and synthetic, non-cellular pharmaceutical drugs, and have varying degrees of success which can depend on the continued administration of the steroid or synthetic drug.

One or more other components can be added to transplanted cells, including selected extracellular matrix components, such as one or more types of collagen known in the art, and/or growth factors, platelet-rich plasma and drugs. Growth factors which can be usefully incorporated into the cell formulation include one or more tissue growth factors known in the art or to be identified in the future, such as but not limited to any member of the TGF-beta family, IGF-I and -II, or growth hormone. Alternatively, the cells of the invention can be genetically engineered to express and produce for growth factors, in conditioned medium. Details on genetic engineering of the cells of the invention are provided in the disclosure and as known to those skilled in the art. Drugs which can be usefully incorporated into the cell formulation include anti-inflammatory compounds, as well as local anesthetics.

### Encapsulation of Extraembryonic Endoderm-Like Cells or Embryonic Stem Cells (ESCs) for Transplantation

Mammalian, primate, or human extraembryonic endoderm-like cells or ESCs, can not be recognized by the immune system or can reduce the immune response as observed in a mixed-lymphocyte reaction.

It is preferred that the extraembryonic endoderm-like cells or ESCs, cells be derived from the patient that is being treated so as to avoid immune rejection. However, where autologous cells are not available, it can be useful to encapsulate the extraembryonic endoderm-like cells or ESCs in a capsule that is permeable to nutrients and oxygen required by the cell and therapeutic factors the cell is secreting such as hormones or erythropoietin, yet impermeable to immune humoral factors and cells. Preferably the encapsulant is hypoallergenic, is easily and stably situated in a target tissue, and provides added protection to the implanted structure.

Protection from immune rejection can also be provided by genetic modification of the extraembryonic endoderm-like cells or ESCs, according to any method known in the art. Autoantibody and CTL resistant cells can be produced using methods such as those disclosed in U.S. Pat. Nos. 5,286,632, 5,320,962, 5,342,761; and in WO 90/11354, WO 92/03917, WO 93/04169, and WO 95/17911. Alternatively, selection of resistant transdifferentiated cells is accomplished by culturing these cells in the presence of autoantibody or IDD associated CTLs or CTLs activated with IDD specific autoantigens. As a result of these techniques, cells having increased resistance to destruction by antibody or T-lymphocyte dependent mechanisms are generated. Such cells can be implanted into an appropriate host in an appropriate tissue as disclosed herein and have increased resistance to destruction by autoimmune processes.

Likewise, the human leukocyte antigen (HLA) profile of the extraembryonic endoderm-like cells or ESCs can be modified, optionally by an iterative process, in which the extraembryonic endoderm-like cells or ESCs are exposed to normal, allogeneic lymphocytes, and surviving cells selected. Alternatively, a site directed mutagenesis approach is used to eliminate the HLA markers from the surface of the extraembryonic endoderm-like cells or ESCs cells, and modified extraembryonic endoderm-like cells or ESCs thereby generated are implanted into a recipient mammal in need of such implantation.

In a specific example, the adeno-associated virus (AAV) vector system carrying the neomycin-resistance gene, neo, is used. AAV can be used to transfect eukaryotic cells (Laface et al. (1988) Virology 162:483). In addition, the pBABE-bleo shuttle vector system carrying the phleomycin-resistance gene is used (Morgenstein et al. (1990) Nucleic Acids Res. 18:3587). This shuttle vector can be used to transform human cells with useful genes as described herein.

Cryopreservation and Banking Extraembryonic Endoderm-Like Cells or Embryonic Stem Cells (ESCs)

Mammalian, primate, or human extraembryonic endoderm-like cells or ESCs of the invention can be cryopreserved and maintained or stored in a “cell bank”. Cryopreservation of cells of the invention can be carried out according to known methods. For example, but not by way of limitation, cells can be suspended in a “freeze medium” such as, for example, culture medium further comprising 0 to 95 percent FBS and 0 to 10 percent dimethylsulfoxide (DMSO), with or without 5 to 10 percent glycerol, at a density, for example, of about 0.5 to 10×10⁶ cells per milliliter. The cells are dispensed into glass or plastic ampoules that are then sealed and transferred to the freezing chamber of a controlled rate freezer. The optimal rate of freezing can be determined empirically. A programmable rate freezer for example, can give a change in temperature of −1 to −10° C. per minute through the heat of fusion can be used. Once the ampoules have reached −180° C., they are transferred to a liquid nitrogen storage area. Cryopreserved cells can be stored for a period of years, though they should be checked at least every 5 years for maintenance of viability.

The cryopreserved cells of the invention constitute a bank of cells, portions of which can be “withdrawn” by thawing and then used as needed. Thawing should generally be carried out rapidly, for example, by transferring an ampoule from liquid nitrogen to a 37° C. water bath. The thawed contents of the ampoule should be immediately transferred under sterile conditions to a culture vessel containing an appropriate medium such as DMEM conditioned with 10 percent FBS.

### Use of Extraembryonic Endoderm-Like Cells for In Vitro Screening of Drug Efficacy or Toxicity

The mammalian, primate, or human extraembryonic endoderm-like cells or ESCs of the invention can be used in vitro to screen a wide variety of compounds for effectiveness and cytotoxicity of pharmaceutical agents, growth/regulatory factors, anti-inflammatory agents. To this end, the cells of the invention, or tissue cultures described above, are maintained in vitro and exposed to the compound to be tested. The activity of a cytotoxic compound can be measured by its ability to damage or kill cells in culture. This can readily be assessed by vital staining techniques. The effect of growth/regulatory factors can be assessed by analyzing the number of living cells in vitro, e.g., by total cell counts, and differential cell counts. This can be accomplished using standard cytological and/or histological techniques, including the use of immunocytochemical techniques employing antibodies that define type-specific cellular antigens. The effect of various drugs on the cells of the invention either in suspension culture or in the three-dimensional system described above can be assessed.

The cells and tissues of the invention can be used as model systems for the study of physiological or pathological conditions. For example. primate or human extraembryonic endoderm-like cells or ESCs of the present invention can be used to study disease states, for example, cancer or neoplastic disease of hematopoietic tissue, autoimmune disease, or genetic diseases.

The cells and tissues of the invention can also be used to study the mechanism of action of cytokines, growth factors, e.g., EPO, and inflammatory mediators, e.g., IL-1, TNF and prostaglandins. In addition, cytotoxic and/or pharmaceutical agents can be screened for those that are most efficacious for a particular patient, such as those that reverse, reduce or prevent cancer or hematopoietic disease, or otherwise enhance the balanced growth of hematopoietic tissue. Agents that prove to be efficacious in vitro could then be used to treat the patient therapeutically.

### Use of Extraembryonic Endoderm-Like Cells to Produce Biological Molecules

In a further aspect, the mammalian, primate, or human extraembryonic endoderm-like cells or ESCs of the invention can be cultured in vitro to produce biological products in high yield. For example, such cells, which either naturally produce a particular biological product of interest (e.g., a growth factor, regulatory factor, or peptide hormone), or have been genetically engineered to produce a biological product, could be clonally expanded using, for example, the three-dimensional culture system described above. If the cells excrete the biological product into the nutrient medium, the product can be readily isolated from the spent or conditioned medium using standard separation techniques, e.g., such as differential protein precipitation, ion-exchange chromatography, gel filtration chromatography, electrophoresis, and HPLC, to name but a few. A “bioreactor” can be used to take advantage of the flow method for feeding, for example, a three-dimensional culture in vitro.

Essentially, as fresh media is passed through the three-dimensional culture, the biological product is washed out of the culture and can then be isolated from the outflow, as above.

Alternatively, a biological product of interest can remain within the cell and, thus, its collection can require that the cells are lysed. The biological product can then be purified using any one or more of the above-listed techniques.

### Methods of Administration

In the methods described herein, the therapeutically effective amount of mammalian, primate, or human extraembryonic endoderm-like cells or ESCs can range from the maximum number of cells that is safely received by the subject to the minimum number of cells necessary for treatment of cancer or neoplastic disease of hematopoietic tissue, autoimmune disease, or genetic diseases. Generally, the therapeutically effective amount of each mammalian, primate, or human extraembryonic endoderm-like cells or ESCs is at least 1×10⁴ per kg of body weight of the subject and, most generally, need not be more than 7×10⁵ of each type of cell per kg. Although it is preferable that the mammalian, primate, or human extraembryonic endoderm-like cells or ESCs are autologous or HLA-compatible with the subject, the mammalian, primate, or human extraembryonic endoderm-like cells or ESCs can be isolated from other individuals or species or from genetically-engineered inbred donor strains, or from in vitro cell cultures.

The therapeutically effective amount of the mammalian, primate, or human extraembryonic endoderm-like cells or ESCs can be suspended in a pharmaceutically acceptable carrier or excipient. Such a carrier includes but is not limited to basal culture medium plus 1% serum albumin, saline, buffered saline, dextrose, water, and combinations thereof. The formulation should suit the mode of administration. Accordingly, the invention provides a use of human hematopoietic tissue producing mammalian, primate, or human extraembryonic endoderm-like cells or ESCs for the manufacture of a medicament to treat an hematopoietic disease in a subject in need thereof. In some aspects, the medicament further comprises recombinant polypeptides, such as growth factors, chemokines or cytokines. In further aspects, the medicaments comprise mammalian, primate, or human extraembryonic endoderm-like cells or ESCs. The cells used to manufacture the medicaments can be isolated, derived, or enriched using any of the variations provided for the methods described herein.

The mammalian, primate, or human extraembryonic endoderm-like cells or ESCs preparation or composition is formulated in accordance with routine procedures as a pharmaceutical composition adapted for intravenous administration to human beings. Typically, compositions for intravenous, intra-arterial administration or administration within the hematopoietic tissue, are solutions in sterile isotonic aqueous buffer. Where necessary, the composition can also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. When the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Pharmaceutically acceptable carriers are determined in part by the particular composition being administered, as well as by the particular method used to administer the composition. Accordingly, there is a wide variety of suitable formulations of pharmaceutical compositions (see, e.g., Alfonso R Gennaro (ed), Remington: The Science and Practice of Pharmacy, formerly Remington's Pharmaceutical Sciences 20th ed., Lippincott, Williams & Wilkins, 2003, incorporated herein by reference in its entirety). The pharmaceutical compositions generally comprise mammalian, primate, or human extraembryonic endoderm-like cells or ESCs in a form suitable for administration to a patient. The pharmaceutical compositions are generally formulated as sterile, substantially isotonic and in full compliance with all Good Manufacturing Practice (GMP) regulations of the U.S. Food and Drug Administration.

A variety of means for administering cells to subjects will, in view of this specification, be apparent to those of skill in the art. Such methods include injection of the cells into a target site in a subject. Cells can be inserted into a delivery device which facilitates introduction by injection or implantation into the subjects. Such delivery devices can include tubes, e.g., catheters, for injecting cells and fluids into the body of a recipient subject. In a preferred aspect, the tubes additionally have a needle, e.g., a syringe, through which the cells of the invention can be introduced into the subject at a desired location. In a preferred aspect, mammalian, primate, or human extraembryonic endoderm-like cells or ESCs are formulated for administration into a blood vessel via a catheter (where the term “catheter” is intended to include any of the various tube-like systems for delivery of substances to a blood vessel). The cells can be prepared for delivery in a variety of different forms. For example, the cells can be suspended in a solution or gel. Cells can be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like.

Modes of administration of the mammalian, primate, or human extraembryonic endoderm-like cells or ESCs, include but are not limited to systemic, intra-organ, intravenous or intra-arterial injection and injection directly into the tissue at the intended site of activity. The preparation can be administered by any convenient route, for example by infusion or bolus injection and can be administered together with other biologically active agents. Administration is preferably systemic. Most preferably, the site of administration is close to or nearest the intended site of activity. In cases when a subject suffers from global ischemia, a systemic administration, such as intravenous administration, is preferred. Without intending to be bound by mechanism, mammalian, primate, or human extraembryonic endoderm-like cells or ESCs, will, when administered, migrate or home to hematopoietic tissue, in response to chemotactic factors produced due to the injury. Ischemic tissue that can be treated by the methods of the invention include, but are not limited to, hematopoietic disease.

The methods described herein, provide a recombinant polypeptide or a drug is administered to the subject in combination with the administration of cells. The polypeptide or drug can be administered to the subject before, concurrently, or after the administration of the cells. In one preferred aspect, the recombinant polypeptide or drug promotes angiogenesis, vasculogenesis, or both. In another aspect, the recombinant polypeptide or drug promotes the proliferation or differentiation of the mammalian, primate, or human extraembryonic endoderm-like cells or ESCs. In one aspect, the recombinant polypeptide is VEGF, BFGF, SDF, CXCR-4 or CXCR-5, or a fragment thereof which retains a therapeutic activity to the ischemic tissue.

In particular, the invention methods are useful for therapeutic treatment of hematopoietic disease in humans. Administration of primate or human extraembryonic endoderm-like cells or ESCs, according to invention methods can be used as a sole treatment or as an adjunct to surgical and/or medical treatment modalities. For example, the methods described herein for treatment of cancer or neoplastic disease of hematopoietic tissue, autoimmune disease, or genetic diseases.

The therapeutically effective amount of the mammalian, primate, or human extraembryonic endoderm-like cells or ESCs, is a maximum number of cells that is safely received by the subject. Because the preferred injection route is intravenous to populate the hematopoietic tissue, the maximum dose should take into consideration the size of the vessels into which the cells are infused, so that the vessels do not become congested or plugged. The minimum number of cells necessary for induction of new blood vessel formation in the hematopoietic tissue can be determined empirically, without undue experimentation, by dose escalation studies. For example, such a dose escalation could begin with approximately 10⁴/kg body weight of primate or human extraembryonic endoderm-like cells or ESCs.

One aspect of the invention further provides a pharmaceutical formulation, comprising: (a) mammalian, primate, or human extraembryonic endoderm-like cells or ESCs, and a pharmaceutically acceptable carrier. In some aspects, the formulation comprises from 10⁴ to 10⁹ mammalian, primate, or human extraembryonic endoderm-like cells or ESCs. In a further aspect, the formulation is prepared for administration by a catheter.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

The following isolated cell lines, an isolated mammalian extraembryonic endoderm-like cell line or primitive endoderm-like cell line, described in the specification and further described in the examples below and designated as PEL P7 cell line have been deposited with the American Type Culture Collection, 10801 University Boulevard, Manassas, Va. 20110-2209 under the Budapest Treaty on Dec. 18, 2006. The isolated mammalian extraembryonic endoderm-like cell line has the ATCC Accession No. indicated: ______.

Other aspects and uses will be apparent to one skilled in the art in light of the present disclosures.

### EXEMPLARY ASPECTS

### EXAMPLE 1

### Human Embryonic Stem Cells (hESCs) Generate a Subpopulation of Migratory Cells that Express Markers of Extraembryonic Endoderm

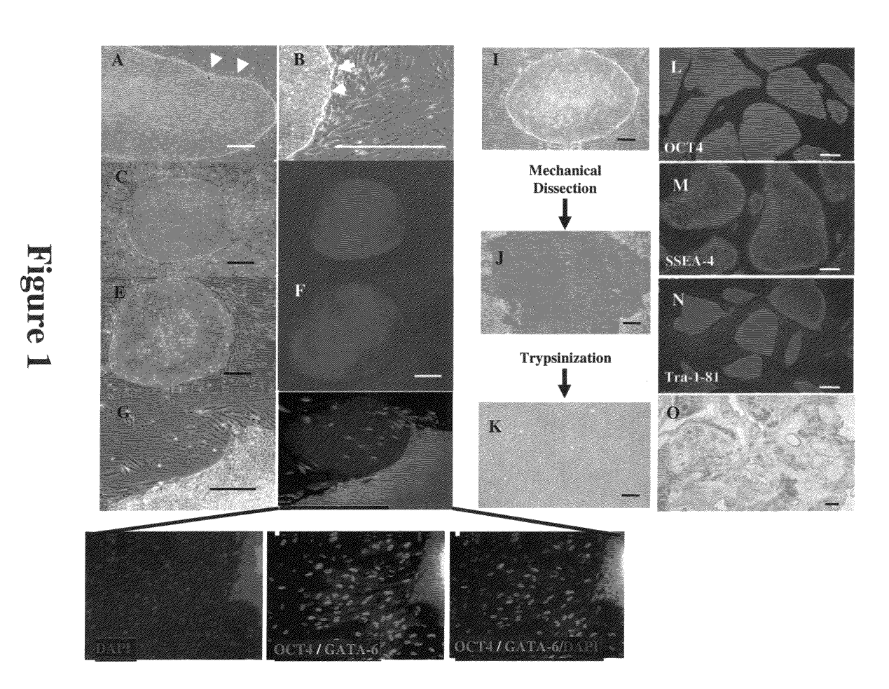

Human embryonic stem cells (hESCs) have been conventionally derived and maintained in serum-containing medium on feeder layers of mitotically inactivated mouse or human fibroblasts, and have been shown to retain pluripotence without feeder layers when they are cultured in the presence of feeder cell-conditioned medium and cocktails of exogenous growth factors. Xu et al., Nat Biotechnol 19:971, 2001; Amit et al., Biol Reprod 70:837, 2004; Li et al., Biotechnol Bioeng 91:688, 2005; Lu et al., Proc Natl Acad Sci USA 103:5688, 2006; Yao et al., Proc Natl Acad Sci USA 103:6907, 2006. In the process of developing a simplified, defined, feeder-free culture system, we cultured hESC on extracellular matrix and observed that they not only formed the tight, smooth-edged colonies typical of undifferentiated cells, but also spontaneously generated a population of cells that migrated away from the colonies (Xu et al., Nat Biotechnol 19:971, 2001; Rosler et al., Dev Dyn 229:259, 2004) [FIGS. 1A,B]. These cells could have arisen from a pre-existing subpopulation that was harbored within the hESC colonies, or they could be newly derived from undifferentiated hESC in response to the culture conditions. To determine the origin of the migratory cells we cultured a clonal hESC population that expressed enhanced green fluorescent protein (eGFP) under control of a ubiquitous promoter, in both feeder-free and mouse embryonic fibroblast (MEF) feeder-containing cultures. FIG. 1 shows that the migratory population expressed eGFP [FIGS. 1 C,D], confirming their clonal origin from the undifferentiated hESC and suggesting that their differentiation was inhibited by the presence of a pre-existing mouse fibroblast feeder layer [FIGS. 1 E,F].

Immunocytochemical analysis showed that the colonies of undifferentiated cells and the clonally-related migratory population expressed distinct markers. The compact colonies were positive for POU5F1/OCT4, a marker of undifferentiated cells [FIGS. 1 G,H]. In contrast, the migratory subpopulation down-regulated expression of POU5F1/OCT4, and was uniformly positive for GATA-6 [FIGS. 1 G,H], which is a marker associated with the primitive endodermal lineage in mouse embryos. Koutsourakis and Langeveld, Development 126:723, 1999. This result suggested that the cells might be similar to the primitive endoderm that is the first differentiated derivative of the ICM during embryogenesis. Gardner and Rossant, J Embryol Exp Morphol 52:141, 1979. On the basis of expression of GATA6, we tentatively termed this cell population PEL (primitive-endoderm-like) cells. However, GATA6 expression is not exclusive to primitive endoderm and is known to be expressed in a variety of adult cell types. Morrisey et al., Dev Biol 177:309, 1996. Therefore, to examine the PEL cells for other markers of extraembryonic endoderm, we isolated a homogeneous population of PEL cells using a sequential mechanical-enzymatic isolation procedure [FIG. 1 I,K] and characterized them by whole genome expression analysis.

FIG. 1 shows primitive endoderm-like cells from undifferentiated hESCs. Typical morphology of an undifferentiated hESC colony in feeder-free defined culture conditions [A] and an enlarged view of a similar colony [B] show the appearance of elongated cells beyond the edge (white arrowheads) of the colonies. Phase contrast [C] and fluorescent [D] images of a clonal eGFP-hESC colony cultured without a feeder layer show that the migratory cells are also GFP-positive and therefore derived from the hESC colony. Phase contrast [E] and fluorescent [F] images of an hESC colony cultured on mouse embryonic fibroblast (MEF) feeder layers show that no GFP-positive fibroblastic cells are generated when a feeder layer is already present. Phase contrast image [G] of an hESC colony and the same field viewed with fluorescence [H] shows that cells within the colonies are positive for POU5F1/OCT4 (green), while cells beyond the periphery show nuclear staining for the primitive endoderm marker GATA-6 (red). In Panels H₁ H₂, and H₃, cells migrating from the hESC colony are shown at higher magnification and counterstained with Dapi to show the nuclear localization of GATA-6 and their immunonegativity for Oct-4; [H₁, Dapi]; [H₂, GATA-6 & Oct-4 dual staining]; [H₃, GATA-6, Oct-4, Dapi triple staining]. PEL cells were isolated after outgrowth from undifferentiated hESC colonies. [I] Colonies are mechanically removed [J] and the remaining cells passaged by trypsinization [K]. hESCs co-cultured with irradiated PEL cells remain undifferentiated, as shown by their expression of POU5F1/OCT4 [L], SSEA-4 [M], and Tra-1-81 [N] in vitro and the hESCs give rise to teratomas in vivo when injected into SCID mice (O). Scale bars: [A, C-F, I, J, K], 50 μm; [B,G,H], 25 μm; [L, M,N], 100 μm; [O], 200 μm.

Table 1 shows the results of gene expression analysis (Illumina Sentrix Human-6 48K BeadChip Arrays) comparing undifferentiated hESCs, hESC-derived PEL cells, and human foreskin fibroblasts (HFFs) (line HS27: ATCC). Compared to hESCs, PEL cells had significantly lower expression of genes associated with undifferentiated hESCs, including POU5F1/OCT4, LIN28, EBAF, UTF1, and ZFP42/REX1. Brandenberger et al., BMC Dev Biol 4:10, 2004; Liu et al., BMC Dev Biol 6:20, 2006; Sato et al., Dev Biol 260:404, 2003. DNMT3B, a DNA methylransferase that establishes new DNA methylation patterns during development (Okano et al., Cell 99:247, 1999), was highly expressed in hESCs, but was down-regulated considerably in the PEL cells after differentiation. The PEL cells expressed higher levels of transcripts associated with extraembryonic endoderm such as GATA6, DAB2, osteonectin, and plasminogen activators PLAT and PLAU. Yamanaka et al, Dev Dyn 9:2301, 2006. The PEL cells also highly expressed the growth factor Inhibin beta A (Activin A), which was only slightly detectable in undifferentiated hESCs. The hESCs differentially expressed the Activin A receptor ACVR2B, indicating that they might be responsive to the Inhibin beta A (Activin A) produced by the PEL cells.

TABLE 1
Relative expression of genes associated with undifferentiated hESCs and extraembryonic endoderm

| | | | | | | |
|---|---|---|---|---|---|---|
| | | | Undifferentiated | | HS27 | |
| Symbol | Accession | Definition | HES | PEL | Fibroblast | Reference |

EMBRYONIC STEM CELL MARKERS

| | | | | | | |
|---|---|---|---|---|---|---|
| ACVR2B | NM_001106.2 | Activin A receptor, type IIB | 1344 | ND | ND | 17 |
| CDH1 | NM_004360.2 | Cadherin 1, type 1, E-cadherin (epithelial) | 7893 | ND | ND | 17 |
| DNMT3B | NM_175849.1 | DNA (cytosine-5-)-methyltransferase 3 beta | 14880 | 60 | 56 | 17 |
| EBAF | NM_003240.2 | Endometrial bleeding associated factor (left-right determination, | 158 | ND | ND | 17 |
| | | factor A; transforming growth factor beta superfamily) | | | | |
| LIN28 | NM_024674.3 | Lin-28 homolog (C. elegans) | 10339 | ND | ND | 17 |
| POU5F1 | NM_002701.2 | POU domain, class 5, transcription factor 1 (OCT4) | 635 | ND | ND | 17 |
| UTF1 | NM_003577.1 | Undifferentiated embryonic cell transcription factor 1 | 2361 | 186 | 148 | 17 |
| ZFP42 | NM_174900.2 | Zinc finger protein 42 | 2678 | ND | ND | 17 |

EXTRAEMBRYONIC ENDODERM MARKERS (PRIMITIVE, PARIETAL AND VISCERAL ENDODERM)

| | | | | | | |
|---|---|---|---|---|---|---|
| ACVR1 | NM_001105.2 | Activin A receptor, type I | 790 | 1990 | 1351 | 3 |
| DAB2 | NM_001343.1 | Disabled homolog 2, mitogen-responsive phosphoprotein | 267 | 6848 | 10304 | 3 |
| FST | NM_013409.1 | Follistatin, transcript variant FST344 | 788 | 1158 | 2213 | 3 |
| FURIN | NM_002569.2 | Furin (paired basic amino acid cleaving enzyme) | 196 | 892 | 435 | 3 |
| GATA6 | NM_005257.3 | GATA binding protein 6 | ND | 2361 | ND | 3 |
| INHBA | NM_002192.1 | Inhibin, beta A (activin A, activin AB alpha polypeptide) | 151 | 6588 | 179 | 3 |
| KRT18 | NM_000224.2 | Keratin 18, transcript variant 1 | 4587 | 14341 | 6094 | 3 |
| NR2F1 | NM_005654.3 | Nuclear receptor subfamily 2, group F, member 1 (COUPTFI) | ND | 147 | 132 | ⁴³ |
| NR2F2 | NM_021005.2 | Nuclear receptor subfamily 2, group F, member 2 (COUPTFII) | ND | 2878 | 1431 | ⁴³ |
| PDGFRA | NM_006206.2 | Platelet-derived growth factor receptor, alpha polypeptide | ND | 1806 | 1072 | 3 |
| PLAT | NM_000931.2 | Plasminogen activator, tissue, transcript variant 2 | 387 | 2104 | 1022 | 3 |
| PLAU | NM_002658.1 | Plasminogen activator, urokinase | 2826 | 15188 | 7522 | 3 |
| PTHR1 | NM_000316.2 | Parathyroid hormone receptor 1 | 446 | 138 | 204 | 3 |
| SPARC | NM_003118.1 | Secreted protein, acidic, cysteine-rich (osteonectin) | 19609 | 72960 | 61401 | 3 |
| THBD | NM_000361.2 | Thrombomodulin | ND | 1214 | ND | 3 |

MESODERM MARKERS

| | | | | | | |
|---|---|---|---|---|---|---|
| CXCL12 | NM_199168.1 | Chemokine (C—X—C motif) | 1892 | 2291 | 6939 | ⁴⁴ |
| | | ligand 12 (stromal cell-derived factor 1) | | | | |
| CXCR4 | NM_003467.1 | Chemokine (C—X—C motif) receptor 4 | 115 | ND | ND | ⁴⁴ |
| FOXF1 | NM_001451.1 | Forkhead box F1 | ND | ND | 463 | ⁴⁵ |
| KDR | NM_002253.1 | Kinase insert domain receptor (a type III receptor tyrosine kinase) | 196 | 148 | 105 | ⁴⁶ |
| ZIC1 | NM_003412.2 | Zic family member 1 (odd-paired homolog, Drosophila) | ND | ND | 778 | ⁴⁷ |

OTHER MARKERS

| | | | | | | |
|---|---|---|---|---|---|---|
| INS | NM_000207.1 | Insulin | 94 | 97 | 66 | |
| NEFH | NM_021076.2 | Neurofilament, heavy polypeptide 200 kDa | 441 | 268 | 96 | ⁴⁸ |
| NES | NM_006617.1 | Nestin (NES) | 324 | ND | 789 | ⁴⁹ |

Comparisons of transcript levels in undifferentiated hESCs (WA09), extraembryonic endoderm-like derivatives (primitive endoderm-like (PEL) cells), and human foreskin fibroblasts (HFF) (HS27) were made using the Illumina 48K BeadArray. Each replicate independently isolated culture (n = 2) was analyzed on a separate array of approximately 48,000 different transcripts. Values are mean signal (arbitrary units) of approximately 60 signals (beads) for each gene. Expression levels in bold are significantly higher than expression levels in plain text (p < 0.05).
ND = not detectable at 99% confidence level.
The following developmentally regulated genes had no detectable expression in any of the three cell types: AFP, CDX2, CER1, CGB5, CITED1, EOMES, ESRRB, FGF4, FGF8, FOXA2, GATA4, GSC, HHEX, HNF4A, IHH, LHX1, LMX1A, MEOX1, MIXL1, NANOG, NODAL, PTHLH, PTHR2, SNAI1, SOX17, SOX7, SOX2, TCF2, VEGF.

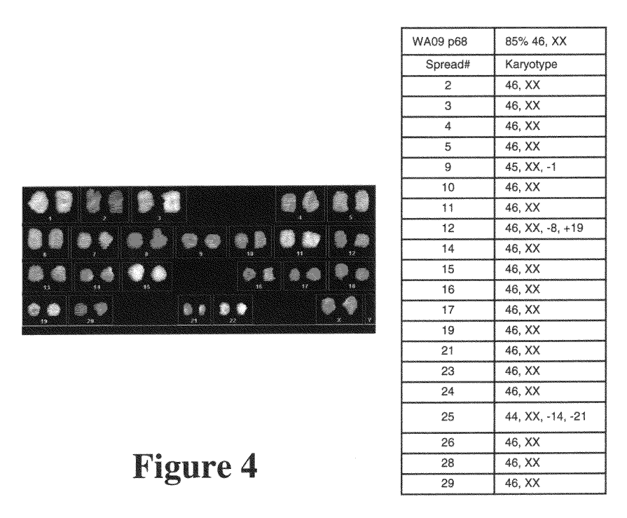

The HFFs were generally similar to the PEL cells in their low expression of hESC-associated genes, and higher expression of markers associated with extraembryonic endoderm, but they did not express detectable levels of GATA6. RT-PCR analysis confirmed the difference in GATA6 expression, showing that only PEL cells expressed this transcription factor. Cdx2, a homeobox transcription factor necessary for trophoblastic development and early embryogenesis (Chawengsaksophak et al., Proc Natl Acad Sci USA 101:7641, 2004; Meissner and Jaenisch, Nature 439:145, 2005) was not detected by gene expression microarray in any of the cell types, and was only slightly detectable by RT-PCR, indicating that none of the cell types expressed key characteristics of trophoblast cells. See FIG. 4.

FIG. 4 shows karyotypic analysis of hESC cultured on mitotically inactivated PEL cells. Karyotyping was performed on hESC cultured for more than 20 passages on PEL cell feeder layers, according to published spectral karyotyping (SKY™) methods. Macville et al., Histochem Cell Biol 108:299, 1997.

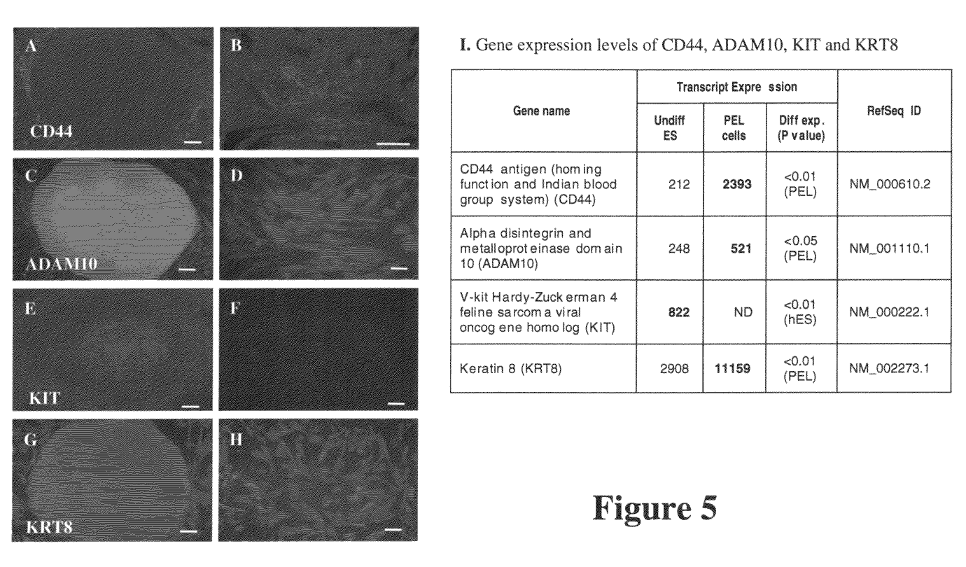

FIG. 5 shows a comparison of markers in hESCs and PEL cells by immunocytochemistry and gene expression microarray. We assayed several developmentally-regulated (Baribault et al., Genes Dev 7:1191, 1993; Campbell et al., Hum Reprod 10:425, 1995; Maretzky et al., Proc Natl Acad Sci USA 102:9182, 2005) genes by both immunocytochemistry and gene expression in the undifferentiated hESC colonies and PEL cells. In each of these cases, the relative gene expression levels and immunocytochemical signals appeared to match. Hyaluronic acid receptor CD44, disintegrin/metalloproteinase ADAM10, and cytokeratin 8 (KRT8) showed higher gene expression and stronger immunocytochemical signal in PEL cells, while the KIT oncogene expression and immunocytochemical signal was higher in the hESCs. [A-H] Immunocytochemical analysis: A, C, E, G show undifferentiated hESCs within colonies and the hESC cell-derived PEL cells at the periphery of the colonies. B, D, F, H show isolated PEL cell lines. An undifferentiated hESC colony is negative for CD44 [A], in contrast to the positive PEL cells at the colony's periphery and in isolated PEL cells [B]. hESCs were strongly positive for ADAM10 while peripheral cells [C] and isolated PEL cells [D] were weakly positive. hESCs were moderately positive for KIT [E] while peripheral cells and isolated PEL cells [F] were negative. Both cells types were positive for cytokeratin 8 [G,H]. [I] Gene expression levels: The levels of expression of the markers tested by immunocytochemistry in A-H were assayed by gene expression microarray. Comparisons of transcript levels in undifferentiated hESCs (WA09) and extraembryonic endoderm-like derivatives (primitive endoderm-like (PEL) cells were made using the Illumina 48K BeadArray. Each replicate independently isolated culture (n=2) was analyzed on a separate array of approximately 48,000 different transcripts. Values are mean signal (arbitrary units) of approximately 60 signals (beads) for each gene. Expression levels in bold are significantly higher than expression levels in plain text (p<0.05). ND=not detectable at 99% confidence level. Scale bars: [A-H], 50 μm.

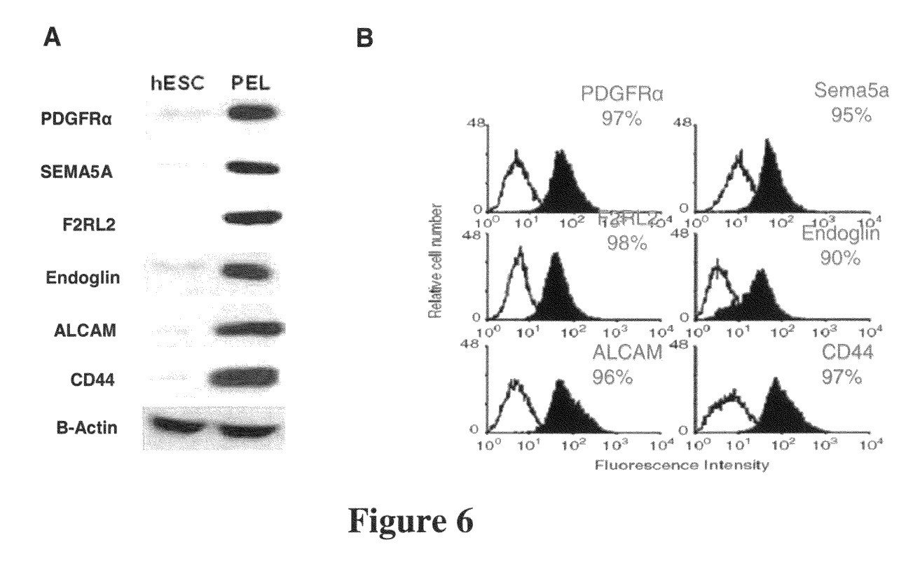

FIG. 6A shows a Western blot analysis of cell lysates obtained from hESC and PEL cells, indicated at the protein level that PEL cells expressed higher levels of PDGRA, SEMA5A, Endoglin, ALCAM and CD44 proteins compared to hESCs. FIG. 6B shows FACS Analysis of PEL cells for PDGRA, SEMA5A, Endoglin, ALCAM and CD44 proteins expression indicated that most (>90%) of the cells express these proteins on surface.

### EXAMPLE 2

### Differentiation of PEL Cells into Visceral Endoderm

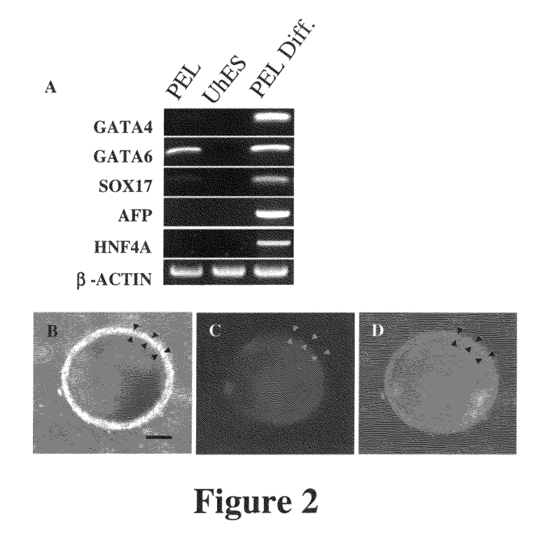

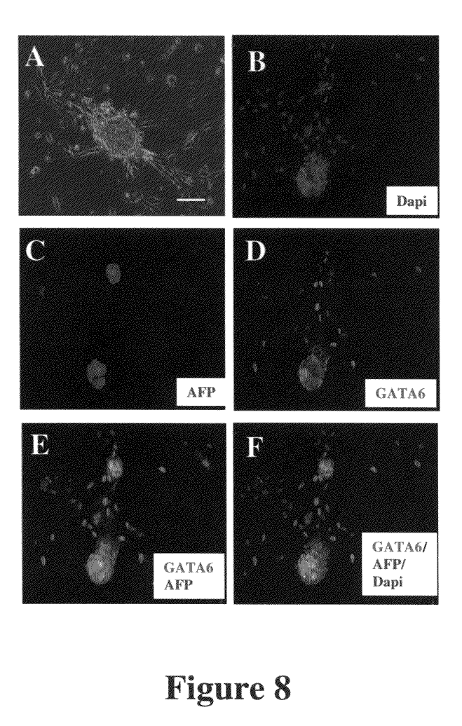

In vivo, a characteristic of primitive endoderm is that it gives rise to extraembryonic visceral endoderm, which is essential for nutrient transport and induction of patterning during embryogenesis. Rossant, Semin Cell Dev Biol. 15:573, 2004; Beddington and Robertson, Cell 96:195, 1999; Ang and Constam, Semin Cell Dev Biol 15:555, 2004. We tested the ability of PEL cells to differentiate into visceral endoderm in vitro by culturing them in aggregates on non-adhesive substrata, in a method similar to that used to test the differentiative abilities of hESC through embryoid body formation. Itskovitz et al., Mol Med 6:88, 2000; Coucouvanis and Martin, Development 126:535, 1999. After several days in aggregate culture, the PEL cells began to express a group of visceral endoderm-associated markers, including alpha fetoprotein (AFP) (Dziadek and Adamson, J Embryol Exp Morphol 43:289, 1978), hepatocyte nuclear factor 4 alpha (HNF4A) (Duncan et al., Proc Natl Acad Sci USA 91:7598, 1994), SOX17 (Kanai-Azuma et al., Development 129:2367, 2002) and GATA4 (Arceci et al., Mol Cell Biol 13:2235, 1993) [FIG. 2A; FIG. 8]. This suggested that the PEL cells could act as precursors to visceral endoderm, a function that is consistent with a primitive endoderm identity.

Another characteristic of primitive endoderm is their behavior in vivo; primitive endoderm cells originate as scattered cells in the ICM and sort out from the population to form an epithelial layer that covers the blastocoelic surface of the ICM. Yamanaka et al., Dev Dyn 9:2301, 2006. During differentiation of embryoid bodies from ESC in vitro, the extraembryonic endoderm typically forms a layer on the outside of the aggregates. Coucouvanis and Martin, Development 126:535, 1999. We used an in vitro assay to ask whether interspersed PEL cells and hESCs would become distributed in a similar manner. We made co-aggregates of eGFP-labeled hESCs and unlabeled PEL cells, and cultured them on non-adhesive substrata. Strikingly, after several days the PEL cells separated from the hESCs and formed a layer covering the outside of the aggregates [FIG. 2B]. This behavior is exactly what one would expect of extraembryonic endoderm cells and serves as additional evidence that the PEL cells possess many of the characteristics of extraembryonic endoderm.

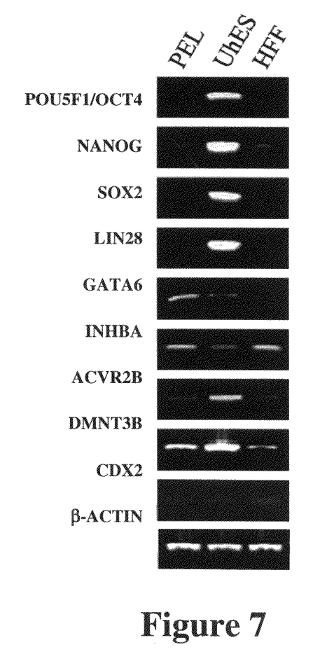

FIG. 7 shows RT-PCR analysis of PEL, hESC, and HFF. Compared to undifferentiated hESCs (“UhES”), PEL cells (“PEL”) expressed higher levels of GATA6 and Inhibin beta A (INHBA), and lower levels of Activin receptor ActRIIB and DNA methylase DNMT3B. PEL cells did not express detectable POU5F1/OCT4, NANOG, SOX2, or LIN28, while undifferentiated hESCs did express these transcripts. Neither cell line expressed CDX2. A human foreskin fibroblast cell line (“HFF”) (Hs27 line from ATCC) shows the same expression pattern as the PEL cells but does not express GATA-6. β-Actin serves as a positive loading control. Transcripts analyzed by PCR were:

| | | |
|---|---|---|
| INHBA | NM_002192.1 | Inhibin, beta A (activin A, activin AB |
| | | alpha polypeptide) |
| POU5F1 | NM_002701.2 | POU domain, class 5, transcription |
| | | factor 1 (OCT4) |
| NANOG | NM_024865.1 | Nanog homeobox (NANOG) |
| SOX2 | NM_003106.2 | SRY (sex determining |
| | | region Y)-box 2 (SOX2) |
| LIN28 | NM_024674.3 | Lin-28 homolog (C. elegans) |
| GATA6 | NM_005257.3 | GATA binding protein 6 |
| INHBA | NM_002192.1 | Inhibin, beta A (activin A, activin AB |
| | | alpha polypeptide) |
| ACVR2B | NM_001106.2 | Activin A receptor, type IIB |
| DNMT3B | NM_175849.1 | DNA (cytosine-5-)-methyltransferase 3 beta |
| CDX2 | NM_001265.2 | Caudal type homeo box transcription |
| | | factor 2 (CDX2) |

FIG. 8 shows immunocytochemical analysis of AFP in cultures of PEL cells differentiated into visceral endoderm. [A] Phase contrast micrograph of a representative aggregate of PEL cells differentiated in suspension and then allowed to attach to plastic. [B-F] Different aggregate cultured under similar conditions and prepared for immunocytochemical analysis. Prior to differentiation, all PEL cells are GATA6-positive but AFP-negative. Following differentiation towards visceral endoderm, AFP-immunopositive cells begin to emerge from the PEL aggregates. [B] DAPI [C] AFP [D] GATA6 [E] GATA6/AFP [F] Merged image of DAPI, AFP and GATA6 immunocytochemical staining. Scale bar in [A]=50 μm

### EXAMPLE 3

### hESC-Derived Primitive-Endoderm-Like (PEL) Cells Support Undifferentiated Growth of hESCs

Since the generation of the PEL cell population appeared to be enhanced in the absence of an exogenous feeder layer, it seemed possible that PEL cells might not only be fulfilling the function of feeder cells in these cultures, but suggested the even more intriguing idea that exogenous feeder cells (such as MEFs) can actually be suboptimal surrogates for the primitive endoderm. hESCs cultured for 20 passages on mitotically inactivated PEL cells remained karyotypically normal [FIG. 4] and continued to express markers associated with the undifferentiated stem cell state, including POU5F1/OCT4, SSEA-4, and Tra-1-81 [FIGS. 1 L-N]. When injected into SCID mice, the hESCs formed teratomas containing derivatives of all three germ layers [FIG. 1 O]. In vitro, when cultured on non-adhesive substrata, the hESCs formed embryoid bodies and differentiated into a variety of cell types, also representing all three germ layers. These results indicate that the PEL cells have the ability to provide long-term maintenance of pluripotent hESC.

### EXAMPLE 4

### PEL Cells Support the Clonal Expansion of Pluripotent hESCs

A variety of methods, including different feeder layers, have been tested in an effort to find conditions that support routine culture of hESCs at clonal density. Amit et al., Dev Biol 227:271, 2000; Pyle et al., Nat Biotechnol 24: 344, 2006. We asked whether PEL cells could support the proliferation of single hESCs by using FACS [FIGS. 2 A, B] to place single eGFP-expressing hESCs into miniwells [FIGS. 2 C, D] with and without irradiated PEL cells. We also tested various serum-free medium combinations containing high bFGF concentrations, the BMP antagonist Noggin, and, in one condition, PEL cell conditioned medium. After 10 days, colonies were detected only in the wells containing PEL cells or PEL-conditioned medium [FIGS. 2 E, F; I]. To confirm that the colonies remained undifferentiated, we showed that they expressed POU5F1/OCT4 [FIGS. 2 G, H], and demonstrated their pluripotence by generating teratomas after transplantation into SCID mice. The cloned cells also retained the ability to form embryoid bodies that spontaneously differentiated in vitro into derivatives of all three germ layers. These results indicate that PEL cells produce factors that support the undifferentiated growth of hESCs at isolated single cell clonal density, and that these factors are secreted into conditioned medium.

FIG. 2 shows the characterization of PEL cells. [A] RT-PCR analysis: PEL cells were further differentiated in vitro into cells resembling visceral endoderm. Differentiated PEL cells (“PEL Diff”) expressed detectable levels of the visceral endoderm markers GATA4, GATA6, SOX17, AFP, and HNF4, while before further differentiation the PEL cells (“PEL”) expressed GATA6 but none of the visceral endoderm-associated markers. None of the markers examined were detected in undifferentiated hESC (“UhES”). β-Actin serves as a positive loading control. Transcripts analyzed by RTPCR:

| Symbol | RefSeq ID | Gene name |
|---|---|---|
| GATA4 | NM_002052.2 | GATA binding protein 4 (GATA4) |
| SOX17 | NM_022454.2 | SRY (sex determining region Y)-box 17 |
| | | (SOX17) |
| GATA6 | NM_005257.3 | GATA binding protein 6 |
| AFP | NM_001134.1 | Alpha-fetoprotein (AFP) |
| HNF4A | NM_178850.1 | Hepatocyte nuclear factor 4, alpha (HNF4A), |
| | | transcript variant 3 |

FIGS. 2[B-D] shows behavior of PEL cells mixed with hESC: Embryoid bodies were formed from unlabeled PEL cells mixed with undifferentiated hESC that expressed eGFP transcribed from an ubiquitous promoter. After 7 days, the unlabeled PEL cells spontaneously sorted out to form a layer outside a core of eGFP-labeled hESC. [B] phase contrast; [C] fluorescence; [D] merged phase contrast and fluorescence. Arrowheads in each panel indicate the unlabeled layer of PEL cells. Scale bar: 50 μm.

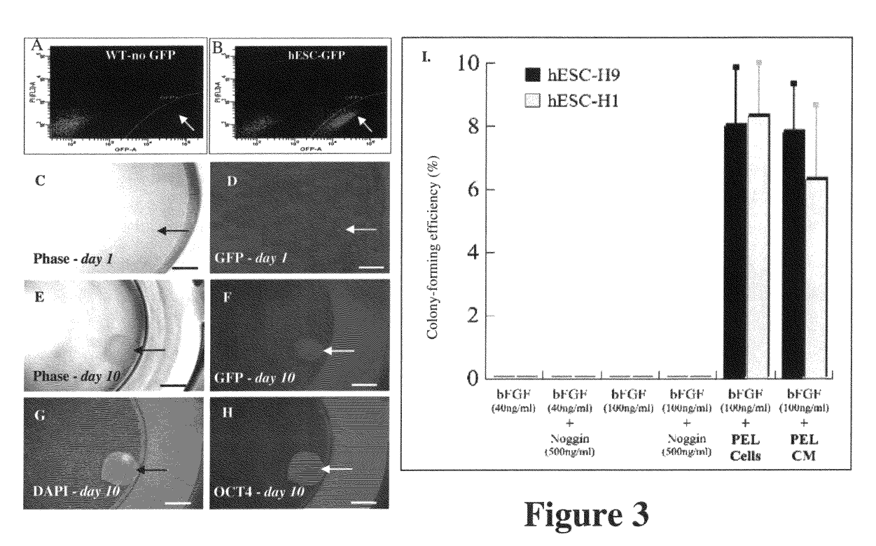

FIG. 3 shows hESC-derived PEL cells support growth of single hESCs into clonal colonies. [A-B] FACS of viable eGFP-expressing hESCs: [A] Negative control showing that wild type (WT) hESCs do not segregate to the GFP-positive bin (arrow); [B] Segregation of eGFP-expressing hESCs to the appropriate GFP bin (arrow). [C-H] Colony formation from a single eGFP-expressing undifferentiated hESC: A single well [C, phase contrast] contains a single eGFP-expressing hESC [D], indicated by arrow at day 1 after seeding. After ten days in culture, the cell has formed an eGFP-expressing colony [E, phase contrast; F, GFP]. Both the hESC and underlying PEL cell feeder layer are shown by DAPI staining of their nuclei [G], but only the cells in the hESC colony are positive for POU5F1/OCT4 [H]. [I] Single eGFP-hESCs were sorted by FACS into Matrigel-coated wells in bFGF in the presence or absence of irradiated PEL cells or PEL cell-conditioned medium, with and without added Noggin. The colony-forming efficiencies under the various conditions indicate that, of the conditions tested, only PEL cells and PEL cell conditioned medium supported expansion of single undifferentiated hESCs. Data are mean values ±S.E. from two separate experiments. Scale bar [C-H]: 100 μm.

### EXAMPLE 5

### Normal Embryonic Stem Cell Lines and PGD-Derived Embryonic Stem Cell Lines Derived Using Non-Immortalized Extraembryonic Endoderm-Like Cells

Preimplantation genetic diagnosis (PGD) is a procedure used to determine karyotypic abnormal embryos in routine in vitro fertilization procedures. These embryos are discarded in normal circumstances, but can be used for derivation of human embryonic stem cells lines. The PGD karyotyping procedure does not perform an exhaustive study of all the mutations present in the genome, selected chromosomal regions which are mutated at a higher frequency are assayed.

Both normal embryonic stem cell lines and PGD-derived embryonic stem cell lines have been derived using non-immortalized extraembryonic endoderm-like cells as described herein. PGDs do not represent a bonafide disease model, as in hESC derived from blastocysts which harbor mutations for example, mutations which cause Down's syndrome Lesch-Nyhan, Huntington's disease, diabetes, cancer, Alzheimer's disease, and such defined diseases. Blastocysts that harbor mutations which cause disease, for example, Down's syndrome or Lesch-Nyhan, cell lines derived from these blastocysts can be used to study these diseases. However, if particular PGD derived hESCs line appears to have particular interesting characteristics such as differentiation into a particular lineage of interest, dopamine neurons or islet cells, it can be used in high throughput applications for drug discovery, screening, toxicology, and in basic science applications to study the mechanism of stem cell division, differentiation and cell death in research laboratories. PGD derived cell lines cannot be used in transplantation therapies.

Thawing and culture of embryos. Frozen 2PN and Day 3 embryos are rapidly thawed and cultured in Blastocyst Culture Media until they develop into blastocysts. Unless they are to be manually dissected (see below), the blastocysts are allowed to hatch, or induced to hatch by applying Acid Tyrode's solution with a micropipette on Day 5 or 6.

Thaw and culture of blastocysts. Embryos that were frozen at the blastocyst stage are thawed and cultured overnight in Blastocyst Culture Medium for reexpansion and hatching.

Preparation of Blastocysts for Culture. there are Several Approaches to Initiating culture of ICMs. The blastocysts can be cultured directly after hatching, subjected to immunosurgery (see Alternative method, below) or manually dissected. Manual dissection is preferred, which appears to improve the viability and attachment in the initial stages of culture. The figure shows a variety of hatched blastocysts. The ICM is indicated by the arrow in each photo.

Dissection of blastocysts to isolate the inner cell mass (ICM). Embryos are placed in Splitting Medium and orientated such that the ICM is towards the Biopsy Pipette with a Biopsy Blade adjacent to it. (See Figure). The ICM can be partially or completely pulled into the Biopsy Pipette. The Biopsy blade is then used to carve away the trophectoderm cells from the ICM, releasing the ICM cells into the pipette.

Preparation of culture dishes. Organ culture dishes, 60 mm dishes with a 10 mm well in the center (otherwise called IVF dishes) are used for the derivations because of the small volume (1 ml) and good optical properties. Small colonies can be visualized in the limited volume of medium, and the shape of the wells makes it possible to dissect colonies with micro tools.

IVF dishes are coated with Matrigel (Becton-Dickinson; growth factor reduced, phenol-free).
Thaw Matrigel on ice to prevent gelling then diluted 1:30 in Knockout DMEM.
Coat center well with 0.5 ml Matrigel solution for 1 hour at room temperature or overnight at 4° C.
Aspirate Matrigel from IVF dishes and add 1 ml of medium to the center well.
Equilibrate the medium in the incubator.

Culture Procedures

Day 1: The embryo is placed into culture.
Release the embryo or dissected ICM from the pipette and place it into Matrigel-coated IVF dish containing 1 ml of medium. Return the dish to the incubator.

Day 2: Feeder cells are added to the dishes in which the ICM was plated.
Feeder cells (mitotically inactivated human fibroblasts) are added to the culture dish without disturbing the embryo. The number of cells added is calculated so that the feeder cells are the same density used for normal hESC culture.
HS27 (human foreskin fibroblasts from ATCC) or hESC-derived fibroblastic primitive endoderm cells were used at a concentration of 50,000-100,000 per IVF dish.
Suspend the appropriate number of cells in about 250 μl and gently add it to the culture dish. If the volume in the dish is already near capacity, remove 250 μl of culture medium from the dish before adding the cells.
Add bFGF to the medium to a final concentration of 20 ng/ml.

Day 3 and forward: Feeding and passaging of embryo/hESC cultures
Growth factors are refreshed every day by adding the same amounts that were present at the original concentrations given in the recipe section.
The remaining factors are added only when fresh medium is added.
Approximately 40% of the culture medium is removed every alternate day and replaced with 50% of fresh culture medium. This discrepancy in volumes is due to the fact that there is always some loss of medium due to evaporation.
Always maintain final concentrations of growth factors as given below in the table.
Typically healthy ICMs should attach within three days Replacing medium for ICMs which have not attached immediately can be done carefully, and is discretionary.

Passaging:
Passage the cultures every 7th day.
Replace medium in the dish with 1 ml fresh ‘complete’ medium.
Mechanically scraped the attached ICM or subcolonies off the IVF dish with sterile insulin syringes while viewing under a 20× objective in a regular microscope. The lower magnification afforded by dissection microscopes can not be adequate to view the smaller colonies.
Transfer the colonies suspended in the fresh medium into a new IVF dish (‘current dish’) with an established feeder layer.
Retain the old dish (“previous dish”)—add 1 ml of fresh “complete” medium. Frequently colonies remain in the dish that housed the previous passage.
Replace both dishes in the incubator.
For subsequent passages colonies from both the previous and current dish are pooled and the previous dish discarded.

Establishing an hESC Line:
Establishing a line is a slow process, and it can be several months before a line is stable. In order for the culture to be designated a cell line it must be successfully frozen and recovered from a frozen stock.
When the population has expanded to at least 20 moderate-sized colonies, cryopreserve 8-10 colonies. A standard hESC freezing protocol was used: or vitrification can be used for cryopreservation.
Maintain the frozen vial for at least a week, then thaw and culture the cells.
If about 80% recovery from freezing is obtained, there is a good chance that a stable line is established.
Continue to expand the cells until several vials can be cryopreserved, then characterize the cells for hESC phenotype.
New hESCs can be tested for the presence of diagnostic markers (SSEA4 and POU5F1/OCT4) by immunofluorescence, and karyotyped as soon as possible.
The differentiation capacity should be tested in vitro and in vivo, and compared to a well-characterized hESC line to obtain a basic comparative profile.
   Culture medium. The medium comprises:
      DMEM/F 12 (with Glutamax)
      βME
      20% Knockout Serum Replacement
      0.1 mM non-essential amino acids
      10 μg/ml gentamicin
      20 ng/ml bFGF
      25 μg/ml bovine insulin
      0.1 μM ascorbic acid
      1× Linoleic acid

In another aspect, in addition to all the above factors N2 and 10 units/ml erythropoietin can be added to the culture medium. N2 supplement (yields final concentrations of 25 μg/ml insulin, 100 μg/ml transferrin, 100 μM putrescine, 30 nM sodium selenite, and 20 nM progesterone).

### EXAMPLE 6

### Protein Composition of hESC, PEL Cells and PEL Cell-Conditioned Medium

To further characterize the changes that occur upon differentiation of PEL cells, and to determine what PEL cell-secreted factors might support hESCs, we used a global proteomic profiling approach, MudPIT (Schirmer et al., Science 301:1380, 2003; Washburn et al., Nat Biotechnol 19:242, 2001), to obtain distinct protein profiles for each cell type. One hundred thirty-two proteins were detected in both hESC and PEL cells, and an additional 167 were detected in undifferentiated hESCs but not in PEL cells. PEL cells produced detectable levels of 55 proteins that were not detected in hESCs. Table 2 shows an overview of this analysis; proteins identified from each cell population were categorized by broad biological functions based on gene ontology (http://www.geneontology.org). Even though three times as many proteins were detected in hESCs than PEL cells, the distribution of the proteins' functions was remarkably similar in the two cell types. The only notable difference was that PEL cells expressed a wider variety of cytoskeletal proteins than the undifferentiated hESCs. This difference probably reflects the change in morphology that accompanies the differentiation of PEL cells, which lose the epithelial character of hESCs in colonies and take on the qualities of migratory cells.

TABLE 2
Biological classification of proteins detected in undifferentiated hESCs and PEL cells

| | Detected* in | Detected* only | Detected* only | Percent in | Percent in |
|---|---|---|---|---|---|
| | both cell | in hESC (total | in PEL (total in | Category, | Category, |
| Biological Process | types | in hESC) | PEL) | hESC | PEL |
| DNA processing & transcription | 15 | 33(48) | 7(22) | 16% | 12% |
| RNA processing & translation | 15 | 17(32) | 1(16) | 11% | 9% |
| Protein biosynthesis | 18 | 24(42) | 5(23) | 14% | 12% |
| Protein modification | 16 | 15(31) | 3(19) | 10% | 10% |
| Signal transduction | 6 | 14(20) | 9(15) | 7% | 8% |
| Cell cycle | 2 | 6(8) | 1(3) | 3% | 2% |
| Cytoskeleton and ECM, | 30 | 12(42) | 13(43) | 14% | 23% |
| Adhesion and Motility | | | | | |
| Ubiquitin cycle | 5 | 10(15) | 5(10) | 5% | 5% |
| Energy pathways | 16 | 19(35) | 7(23) | 12% | 12% |
| Other function | 9 | 17(26) | 4(13) | 9% | 7% |

Criteria for detection of a protein were that at least 3 representative peptides were detected (sequence count) and at least 10% of the protein sequence was detected (sequence coverage). Biological processes are as ascribed to the genes by the Gene Ontology database (http://www.geneontology.org) or deduced from the OMIM entries for these genes.

Evidence provided herein that that PEL cell-conditioned medium supported clonal growth of undifferentiated hESCs suggests that proteins involved in maintenance of pluripotence can be secreted into the medium by PEL cells. All of the proteins detected in PEL-conditioned medium have been analyzed to compare the expression levels of the transcripts for these proteins in PEL cells and hESCs. Proteomic analysis of the PEL-conditioned medium showed that 56 proteins were detected; the majority of the proteins were ECM proteins, growth factor related proteins, proteases and protease inhibitors, and cell surface proteins. Interestingly, when we compared the expression levels of the proteins in conditioned medium with the levels of expression of their transcripts in the PEL cells and hESCs, we noted that PEL cells expressed nearly all of the transcripts for these proteins at significantly higher levels than hESCs. The ECM proteins included laminin 1, fibronectin, collagens, and the proteases and protease inhibitors detected are involved in both ECM remodeling and processing of growth factors. Among the cell surface proteins detected were adhesion and cell signaling proteins, including cadherins, chloride intracellular channel 1, and transmembrane receptor PTK7. Proteins detected in the growth factor-related category included DKK3 (Dickkopf-related protein 3) and Inhibin beta A.

### EXAMPLE 7

### hESCs Spontaneously Generate a Clonally-Related Population of Cells That Resemble the Extraembryonic Endoderm

hESCs are considered to be key to future cell therapies, but an important short-term application for these cells is the unprecedented opportunity they offer for understanding the molecular events that control early human embryonic development. We report here an aspect of hESC development in vitro that can reflect some of the earliest developmental events that occur in vivo. When cultured in the absence of exogenous feeder cells, hESCs spontaneously generate a clonally-related population of cells that resemble the extraembryonic endoderm that is generated from the ICM of the blastocyst in the first few days of embryonic development. We termed these cells primitive endoderm-like (“PEL”), because the striking characteristic of their initial differentiation from hESC colonies is the onset of expression of the transcription factor GATA6, which characterizes the primitive endoderm precursors that emerge from mouse ICMs. Our observations suggest that PEL cells can be an in vitro counterpart of this early differentiation event, and thus be a useful model for determining the molecular basis of control of early differentiation in human embryogenesis. The extraembryonic primitive endoderm is the first cellular lineage to emerge in preimplantation embryos, defining the boundaries of the epiblast and possibly playing a role in maintaining the pluripotence of the ICM. The importance of the primitive endoderm to pluripotent cell growth in the early mammalian embryo in vivo is evident in GATA-6 and DAB-2 knock-out mice which have defective primitive endoderm. Morrisey et al., Genes Dev 12:3579, 1998; Yang et al., Dev Biol 251:27, 2002. These null mice develop normally until early egg cylinder stages, but then show reduced proliferation in the epiblast and fail to gastrulate. The GATA 6 −/− blastocysts have normal ICMs and surrounding trophoblast layer, but they lack detectable primitive endoderm. While ICMs from wild type embryos expand in the absence of added feeders, GATA 6 −/− blastocysts attach to the plates but the ICMs fail to grow in the absence of a feeder layer and eventually degenerate. Similarly, DAB-2-deficient mutants are lethal in embryogenesis due to defective cell positioning and formation of the visceral endoderm. In DAB-2 −/− blastocysts in vitro, initially cells with characteristics of endoderm, trophectoderm, and ICM are observed in the outgrowth of the mutant blastocysts. However, the DAB-2−/− extraembryonic endodermal cells are dispersed and disorganized compared to those from wild type blastocysts, the ICMs fail to expand, and the outgrowths degenerate. These reports highlight the importance of the primitive endoderm in maintenance of pluripotent proliferating cells both in vivo and in vitro. PEL cells and their secreted products supported growth of colonies from single isolated hESCs, with greater efficiency than reportedly achieved with primary mouse embryonic fibroblasts (Amit et al., Dev Biol 227:271, 2000) and comparable to the highest cloning efficiencies reported for hESC. Pyle et al., Nat Biotechnol 24: 344, 2006. Among the candidates for hESC support that we identified in the conditioned medium are growth factors, notably members of the insulin-like growth factor and TGF-β pathways. One particularly intriguing detectable component of the PEL conditioned medium is Inhibin beta A, a subunit of the growth factor Activin A, which is an inducer of the TGFβ/nodal signaling pathway and has been independently reported to be supportive of the ICM in vivo and hESC pluripotence in vitro (Beattie et al., Stem Cells 23:489, 2005; James et al., Development 132:1273, 2005; Vallier and Pederson, J Cell Sci 118:4495, 2005); its production by PEL cells can be significant for their effectiveness in the maintenance of hESC self-renewal and pluripotence.

The PEL cells also secrete ECM proteins, proteases, and protease inhibitors, indicating an active process of remodeling of their extracellular environment. The ECM produced by PEL cells can aid in the attachment of single hESCs and expansion of hESC colonies, and enrich the local environment in endogenous and exogenous growth factors. Proteases and protease inhibitors not only modify ECM proteins, but also play critical roles in processing growth factors; they can have a direct regulatory function in signaling by cleaving cell surface receptors. Our identification of the proteins shed into the media by PEL cells can help our understanding of how activating or inactivating signaling events can regulate pluripotence in hESCs.

In summary, we have identified a population of primitive endoderm-like (PEL) cells spontaneously derived from hESCs that efficiently support the proliferation of undifferentiated pluripotent hESCs in vitro. Our results are consistent with developmental studies that suggest that primitive endoderm is critical in vivo for maintenance of a pluripotent ICM in preimplantation human embryos, perhaps to promote controlled expansion of ICM cells that is subsequently receptive to differentiating cues. Since hESCs are an expanded population that is derived from and resembles ICM cells, it seems appropriate that primitive endoderm cells are one of the first populations generated during their growth in vitro. Our results raise the intriguing possibility that mouse and human fibroblasts conventionally used in hESC cultures can owe their effectiveness as feeder layers to their similarities to embryonic primitive endoderm. Preliminary cluster analysis of the whole genome expression data indicates that PEL cells and human foreskin fibroblasts are more similar in gene expression profile to each other than they are to hESCs. One notable exception to this similarity was expression of GATA6, a transcription factor characteristic of the primitive endoderm; this marker was expressed in PEL cells but in not human foreskin fibroblasts or hESCs. We are currently comparing a variety of cell lines used as feeder layers (including MEFs, HFFs, hESC-derived immortalized fibroblasts, and STO fibroblasts) to identify the characteristics shared by all cell types that support undifferentiated hESC growth, a quality which we term “feedemess.” It will also be of great interest to investigate the differences among these cell types to determine what factors correlate with the relative effectiveness of these cell lines to support undifferentiated hESC growth, perhaps allowing us to develop quality control standards for hESC culture systems suitable for clinical use. Not only will further analysis of PEL cells allow refinements in hESC culture conditions that will enhance their use for cell therapy, but further investigations into the nature of PEL-hESC interactions will also contribute to our knowledge of the molecular mechanisms controlling early embryogenesis.

### EXAMPLE 8

### Methods

hESC Culture. hESC lines WA01 (H1) and WA09 (H9) (WiCell, Madison Wis.) were initially maintained on irradiated mouse embryonic fibroblast (MEF) feeder cells in medium that consisted of DMEM/F-12 (80%), Knockout Serum Replacement (20%), L-alanyl-L-glutamine (GlutaMax; 2 mM), MEM nonessential amino acids (1×), β-Mercaptoethanol (100 μM) (all from Invitrogen, Carlsbad, Calif.), and bFGF (4 ng/ml) (PeproTech Inc., Rocky Hill, N.J.) as described previously (Thomson et al., Science 282:1145, 1998), then transferred to human feeder layers (HS27 line, ATCC). For feeder-free growth, cells were transferred to Matrigel (growth factor-reduced, Becton Dickinson, Bedford, Mass.) or human purified laminin-coated dishes, and cultured in the same medium with a higher concentration of bFGF (20 ng/ml). For maintenance of the hESC under defined conditions, the hESC were cultured in a mixture of DMEM/F-12 or KO-DMEM with, L-alanyl-L-glutamine or L-glutamine (2 mM), MEM essential amino acids solution (1×), MEM nonessential amino acids solution (1×), and β-mercaptoethanol (100 μM), bFGF (20 ng/ml), insulin (20 μg/ml), transferrin (8 μg/ml), albumin (10 mg/ml), and ascorbic acid (50 μg/ml) on collagen/laminin combination or on purified human laminin. Only bFGF, insulin, ascorbic acid, and laminin were both sufficient and necessary for permitting and promoting the emergence of PEL cells from undifferentiated hESCs under defined conditions. hESCs were mechanically passaged every 5 to 7 days by cutting undifferentiated hESC colonies into small pieces using a 27 G PrecisionGlide Needle attached to a 1 ml syringe (Becton Dickinson, Bedford, Mass.).

Isolation of hESC-derived PEL cells. WA09 hESC-derived PEL cells were isolated from the differentiated cells surrounding the periphery of undifferentiated hESC colonies grown in feeder-free defined culture. A two-step mechanical/enzymatic treatment method was employed as illustrated in FIGS. 1 I-K. First, all of the morphologically distinct hESC colonies were mechanically dissected away from the cultures. Then the remaining cells were lifted by brief treatment with 0.05% trypsin and then transferred to new Matrigel- or laminin-coated plates containing hESC medium. The PEL cells were further purified by repeating the isolation procedure multiple times until no morphologically hESC-like cells were observed. POU5F1/OCT4 staining confirmed that no positive cells remained. The PEL cells were expanded and cryopreserved. For some experiments, “feeder-like” layers were prepared from PEL cells by irradiation in the same manner as fibroblast cell lines.

PEL Cell Differentiation to Visceral Endoderm. PEL cells were lifted using 0.05% trypsin and then transferred onto 6-well ultra-low attachment plates (Corning, Acton, Mass.) and cultured at 37° C., 5% CO₂ for 7 days in medium that consisted of DMEM/F-12 (80%), Knockout Serum Replacement (20%), L-alanyl-L-glutamine (GlutaMax; 2 mM), MEM nonessential amino acids (1×), β-Mercaptoethanol (100 μM) (all from Invitrogen, Carlsbad, Calif.), bFGF (20 ng/ml) (PeproTech Inc., Rocky Hill, N.J.) and BMP2 (10 ng/ml) (R&D Systems, Minneapolis, Minn.). To create hybrid hESC/PEL embryoid bodies, eGFP-expressing hESCs and unlabeled PEL cells were detached from the tissue culture plate using 0.05% trypsin and then transferred onto low-adherence 6-well plates (Corning) at 5% CO₂ in 4 ml medium containing DMEM/F-12 (80%), Knockout Serum Replacement (20%), L-alanyl-L-glutamine (GlutaMax; 2 mM), MEM nonessential amino acids (1×), β-Mercaptoethanol (100 μM) (all from Invitrogen, Carlsbad, Calif.), and bFGF (20 ng/ml) (PeproTech Inc., Rocky Hill, N.J.).

Derivation of eGFP-expressing hESC. Lentiviral vector pFUGW was generated as described previously. Lois et al., Science 295:868, 2002. Briefly, lentiviral vectors were produced by co-transfecting the transfer vector pFUGW, the HIV-1 packaging vector 8.9, and the VSVG envelope glycoprotein (all gifts from D. Baltimore, California Institute of Technology) into 293 fibroblasts and concentrated as described previously. Undifferentiated hESCs (line WA01 [passage 49] and line WA09 [passage 45]) that had been growing in feeder-free culture for 4 days were incubated with lentiviral vector particles and polybrene (6 μg/ml; Sigma) overnight and the medium was changed the next day. After 7 days of continuous culturing in the defined conditions, hESC colonies that displayed homogenous expression of eGFP were each mechanically picked and individually transferred to wells of 6 well plates. The eGFP-positive undifferentiated hESC subcultures were maintained under the defined culture conditions. For testing growth of colonies from single cells, eGFP-positive colonies were dissociated and sorted by FACS into 96 well plates (see below). Colonies that were observed to be derived from single cells were expanded and characterized.

Fluorescence Activated Cell Sorting (FACS) and single-cell culture. Undifferentiated eGFP-hESCs were dissociated with 0.05% trypsin/0.53 mM EDTA (Invitrogen) into a suspension of single cells and small clusters. Dissociated cells were filtered through 85 μm Nitex mesh to remove aggregates and then sorted on a FACSVantage SE equipped with DiVa electronics and software (Becton Dickinson Biosciences). The GFP signal was excited with an argon laser tuned to 488 nm at 200 mW of power and the emission signal was collected through a 530/30 bandpass filter. The eGFP-positive cells were sorted into wells of a 96 well plate (1 eGFP cell/well) at 15 psi using a 100-μm nozzle tip. Propidium iodide was used to exclude dead cells and only live cells were used for sorting. Cells were sorted into wells without feeder layers, containing bFGF (40 or 100 ng/ml) with or without Noggin (500 ng/ml) in hESC medium, or into wells containing mitotically inactivated PEL cells or PEL cell conditioned medium [48 hours incubation at 37° C. in serum-free medium containing ITS supplement (Invitrogen) and 100 ng/ml bFGF but no serum or serum replacement.]

Illumina Microarray Analysis. RNA was isolated from cultured cells using the Qiagen RNEasy kit (Qiagen, Inc, Valencia, Calif.). Two PEL cultures, 2 undifferentiated hESC (WA09) cultures, and 2 HS27 human foreskin fibroblast (HFF) cultures were harvested separately and served as biological replicates. To assure that only undifferentiated hESCs were isolated, colonies were isolated by hand using a micropipette. Sample preparation and analysis was performed as previously described. Schwartz et al., Stem Cells Dev 14:517, 2005; Cai et al., Stem Cells 24:516, 2006. Briefly, amplification was performed using 100 ng of total RNA using the Illumina RNA Amplification kit (Ambion, Inc., Austin, Tex.) following the manufacturer's instructions; labeling was done by incorporating of biotin-16-UTP (Perkin Elmer Life and Analytical Sciences, Boston, Mass.) present at a ratio of 1:1 with unlabeled UTP. Labeled, amplified material (700 ng per array) was hybridized to the Illumina Sentrix Human 6 BeadChip according to the manufacturer's instructions (Illumina, Inc., San Diego, Calif.). Arrays were washed, and then stained with Amersham fluorolink streptavidin-Cy3 (GE Healthcare Bio-Sciences, Little Chalfont, UK) according to methods provided by the manufacturer. Arrays were scanned with an Illumina BeadArray Reader confocal scanner and array data processing and analysis were performed using Illumina BeadStudio software. The Illumina BeadArrays have an average of 30 beads of each type (50-mer complementary oligonucleotides) in each array, so for each set of biological replicates we obtained approximately 60 independent measurements of hybridization for each transcript. Differential expression of individual genes between groups was calculated by the t-test.

RT-PCR. Expression of several gene transcripts was probed by semiquantitative RT-PCR. Initial denaturation was carried out at 94° C. for 2 minutes, followed by 35 cycles of PCR (94° C. for 30 seconds, 55° C. for 30 seconds, 72° C. for 1 minute). Primers used and their expected products are:

ACTIVIN A (INHIBIN BETA A): product 262 bp,
5′-CTTGAAGAAGAGACCCGAT-3′;
5′-CTTCTGCACGCTCCACTAC-3′;
ACTIVIN RECEPTOR IIB(ACTRIIB-2B): product 556 bp,
5′-ACACGGGAGTGCATCTACTACAACG-3′;
5′-TTCATGAGCTGGGCCTTCCAGACAC-3′;
AFP: product 676 bp,
5′-AGAACCTGTCACAAGCTGTG-3′;
5′-CACAGCAAGCTGAGGATGTC-3′
beta-Actin: product 400 bp,
5′-TGGCACCACACC TTTCTACAATGAGC-3′,
5′-GCACAGCTTCTCCTTAA TGTCACGC-3′;
CDX2: product 563 bp,
5′-GAACCTGTGCGAGTGGATGCG-3′;
5′-GGTCTATGGCTGTGGGTGGGAG-3′;
DNMT3B: product 433 bp,
5′-CTCTTACCTTACCATCGACC-3′,
5′-CTCCAGAGCATGGTACATGG-3′;
GATA4: product 218 bp,
5′-CATCAAGACGGAGCCTGGCC-3′;
5′-TGACTGTCGGCCAAGACCAG-3′;
HNF4: product 762 bp,
5′-GCTTGGTTCTCGTTGAGTGG-3′;
5′-CAGGAGCTTATAGGGGCTCAGAC-3′;
LIN-28: product 420 bp,
5′-AGTAAGCTGCACATGGAAGG-3′;
5′-ATTGTGGCTCAATTCTGTGC-3′;
SOX2: product 370 bp,
5′-CCGCATG TACAACATGATGG-3′;
5′-CTTCTTCATGAGCGTCT TGG-3′;
GATA-6: product 213 bp,
5′-CCATGACTCCAACTTCCACC-3′;
5′-ACGGAGGACGTGACTTCGGC-3′;
NANOG: product 493 bp,
5′-GGCAAACAACCCACTTCTGC-3′,
5′-TGTT CCAGGCCTGATTGTTC-3′;
POU5F1: product 247 bp,
5′-CGTGAAGCTGGAGAAGGAGAAGCTG-3′,
5′-CAAGGGCCGCAGCTTACACATGTTC-3′;
SOX 17: product 181
5′-CGCACGGAATTTGAACAGTA-3′;
5′-GGATCAGGGACCTGTCACAC-3′.

Sample preparation for proteomic analysis. All chemicals were purchased from Sigma-Aldrich unless otherwise noted. Cell pellets of the undifferentiated hESCs (WA09) and the PEL cells (1×10⁶ to 7×10⁷) were washed with 1×PBS and resuspended in 200 μL 1×PBS. Each cell pellet was homogenized with five passages through an insulin syringe and separated into two aliquots. Aliquots from each cell line were prepared by the following methods.

Method 1. An aliquot from each cell line was centrifuged at 18,000×g for 30 min at 4° C. to separate the cell lysate into soluble and insoluble fractions.

Soluble fraction: proteins from the soluble fraction were precipitated with MeOH/CHCl₃, and the protein pellets were resuspended in 50 mM ammonium bicarbonate with 8M Urea.

Insoluble fraction: proteins from the insoluble fraction were first cleaved chemically with CNBr. Briefly, 50 μL CNBr (500 mg/mL in 90% formic acid) were added to the insoluble fractions and incubated in the dark at RT overnight. Next, the reactions were neutralized by gradual addition of 30% ammonium hydroxide and saturated ammonium bicarbonate. Finally, solid urea was added in the solution to 8M.

Alkylation and carboxymethylation: 25 mM of DTT (final concentration) were added to all samples and the reactions were incubated at 50° C. for 45 minutes. Next, 25 mM of iodoacetamide (final concentration) were added to the samples and the reactions were carried out in the dark at room temperature for 45 minutes.

Protein digest in solution: All samples were digested with Endoproteinase Lys C (1:100 enzyme:substrate) (Roche Applied Bioscience, IN) overnight at 37° C. with gentle shaking. Next, the samples were diluted to 2 M urea with 100 mM Tris-HCl, pH 8.5 and 1 mM CaCl₂ (final concentration). 10 μL of porozyme trypsin beads (Perspective Biosystems) were added to the samples and the digest was carried out overnight at 37 C with gentle shaking. The reactions were stopped by the addition of formic acid (5% final concentration).

Method 2. An aliquot from each cell line was separated into soluble and insoluble fractions as described above. Proteins from the soluble fraction were precipitated with MeOH/CHCl₃ and both pellets from the soluble and insoluble fractions were resuspended in 0.2 M Na₂CO₃, pH 11. Then, samples were alkylated and carboxymethylated as described above. Proteinase K (1:100 enzyme/substrate) was added to the samples, and the reactions were incubated at 37° C. for 3 hours. A second aliquot of proteinase K (1:50 enzyme/substrate) was added to the samples and the reactions were carried out an additional 1.5 hours. Formic acid was added to final concentration of 5% to stop the reactions.

Multidimensional chromatography and tandem mass spectrometry. Peptide mixtures were pressure-loaded onto a 250 μm inner diameter (i.d.) fused-silica capillary packed first with 4 cm of 5 μm strong cation exchange material (Partisphere SCX, Whatman), followed by 2 cm of 5 μm C18 reverse phase (RP) particles (Aqua, Phenomenex or Polaris 2000, Metachem Technologies). Loaded and washed microcapillaries were connected via a 2 μm filtered union (UpChurch Scientific) to a 100 μm i.d. column, which had been pulled to a 5 μm i.d. tip using a P-2000 CO₂ laser puller (Sutter Instruments), then packed with 10 cm of RP particles and equilibrated in 5% acetonitrile, 0.1% formic acid (Buffer A). This split-column was then installed in-line with a Quaternary Agilent 1100 series HPLC pump. Overflow tubing was used to decrease the flow rate from 0.1 ml/min to about 200-300 nl/min. Fully automated 12 step chromatography runs were carried out. Three different elution buffers were used: 5% acetonitrile, 0.1% formic acid (Buffer A); 80% acetonitrile, 0.1% formic acid (Buffer B); and 0.5 M ammonium acetate, 5% acetonitrile, 0.1% formic acid (Buffer C). In such sequences of chromatographic events, peptides are sequentially eluted from the SCX resin to the RP resin by increasing salt steps (increase in Buffer C concentration), followed by organic gradients (increase in Buffer B concentration). The last chromatography step consists in a high salt wash with 100% Buffer C followed by acetonitrile gradient. The application of a 2.5 kV distal voltage electrosprayed the eluting peptides directly into an LCQ-Deca ion trap mass spectrometer equipped with a nano-LC electrospray ionization source (ThermoFinnigan). Full MS spectra were recorded on the peptides over a 400 to 1,600 m/z range, followed by three tandem mass (MS/MS) events sequentially generated in a data-dependent manner on the first, second, and third most intense ions selected from the full MS spectrum (at 35% collision energy). Mass spectrometer scan functions and HPLC solvent gradients were controlled by the Xcalibur data system (ThermoFinnigan).

Interpretation of MS/MS datasets. SEQUEST was used to match MS/MS spectra to peptides in a database containing Human sequences downloaded from the human International Protein Index (IPI; www.ensembl.org). To minimize false positive identification, MS/MS spectra generated from each sample were searched against a combined protein database, which includes the IPI data appended with a decoy database generated by reversing the protein sequences from the same database.

The validity of peptide/spectrum matches was hence assessed using the SEQUEST-defined parameters, cross-correlation score (XCorr) and normalized difference in cross-correlation scores (DeltaCn). Spectra/peptide matches were only retained if they had a DeltaCn of at least 0.08 and minimum XCorr of 1.8 for +1, 2.5 for +2, and 3.5 for +3 spectra. A minimum sequence length of 7 amino acid residues was required. In addition, a 5% false positive rate was used to filter the protein list. A modified version of the DTASelect (Tab et al., J Proteome Res 1:21, 2002) was used to select and sort peptide/spectrum matches passing this criteria set. Peptide hits from multiple runs were compared using CONTRAST. Tab et al., J Proteome Res 1:21, 2002. Cellular proteins were considered to be detected if they were identified by at least 2 spectra passing all of the selection criteria with at least 10% sequence coverage. For the culture media, which contained less protein, we set the detection limit at 5% coverage with the same spectra requirements.

Immunocytochemistry. Cultures were fixed with 4% paraformaldehyde and blocked in 1×PBS containing 0.2% Triton X-100 and 2% BSA. The cells were incubated with the primary antibody in 0.1% Triton X-100 in PBS at 4° C. overnight. Then, secondary antibody (Invitrogen) was added and incubated at RT for 45 min. After staining with DAPI, cells were visualized with a fluorescence microscope. Primary antibody to AFP, GATA6, POU51/OCT4, SSEA-4, and Tra-1-81 were obtained from Santa Cruz Biotechnology. CD44, ADAM10, Keratin 8, and KIT antibodies were obtained from Chemicon International.

Teratoma formation. Approximately 10⁴ hESCs were injected beneath the kidney capsule of adult male Severe Combined Immunodeficient (SCID) mice. After 21 to 90 days, mice were sacrificed and teratomas were dissected, fixed in Bouin's fixative overnight, processed for paraffin sections and stained with hematoxylin and eosin. Sections were examined for evidence of tissue differentiation using bright field light microscopy and photographed as appropriate. All procedures involving mice were carried out in accordance with Institutional and NIH guidelines.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed:

1. An isolated mammalian non-immortalized extraembryonic endoderm-like cell line or variant cell line thereof.

2. The extraembryonic endoderm-like cell line of claim 1 comprising a gene and protein expression profile having decreased expression of genes associated with undifferentiated mammalian embryonic stem cells and increased expression of genes associated with extraembryonic endoderm.

3. The extraembryonic endoderm-like cell line of claim 2 wherein the gene and protein expression profile of the isolated extraembryonic endoderm-like cell line decreases expression of genes POU5F1/Oct4, LIN28, DNMT3B, ZIC2, ZIC3, and UTFI, and increases expression of genes GATA6, DAB-2, basement membrane genes, laminin (LAMC1), collagens, fibronectin (FN1), and nidogens, compared to mammalian embryonic stem cells.

4. A cell conditioned medium derived from growth of an isolated mammalian extraembryonic endoderm-like cell line.

5. An isolated cell population obtained by differentiating primate pluripotent stem cells, in which at least 5% of the cells express a gene or protein expression profile having decreased expression of one or more of POU5F1/Oct4, LIN28, DNMT3B, ZIC2, ZIC3, and UTF1, and having increased expression one or more of GATA6, DAB-2, basement membrane genes, laminin (LAMC1), collagens, fibronectin (FN1), and nidogens.

6. The isolated cell population of claim 5 wherein at least 5% of the cells express at least two of the following markers: GATA6, DAB-2, basement membrane genes, laminin (LAMC1), collagens, fibronectin (FN1), and nidogens.

7. The isolated cell population of claim 5, comprising less than 1% undifferentiated pluripotent stem cells.

8. The isolated cell population of claim 5, wherein the pluripotent stem cells are embryonic stem cells.

9. The isolated cell population of claim 8, wherein the embryonic stem cells are human embryonic stem cells.

10. The isolated cell population of claim 8, wherein the isolated cell population is extraembryonic-endoderm-like cells.

11. The isolated cell population of claim 10, wherein the isolated cell population is a primate extraembryonic-endoderm-like cell population.

12. The isolated cell population of claim 11, wherein the isolated cell population is a human extraembryonic-endoderm-like cell population.

13. The isolated cell population of claim 5, wherein the isolated cell population is primitive endoderm, parietal endoderm, or visceral endoderm.

14. An isolated mammalian extraembryonic endoderm-like cell line deposited as ATCC Accession Number ______.

15. A set of at least two isolated cell populations consisting of: a first cell population comprising one or more primate pluripotent stem cells isolated from a primate preimplantation primate embryo or cells thereof, and a second cell population that proliferates in culture, comprising at least 30% pluripotent stem cell-derived extraembryonic endoderm-like cells, identifiable by a criteria that the extraembryonic endoderm-like cells express one or more of the following: GATA6, DAB-2, basement membrane genes, laminin (LAMC1), collagens, fibronectin (FN1), or nidogens.

16. The set of two isolated cell populations of claim 15 wherein the first cell population is isolated from the primate preimplantation primate embryo or cells thereof having a normal or non-disease state.

17. The set of two isolated cell populations of claim 15 wherein the first cell population is isolated from the primate preimplantation primate embryo having a disease state.

18. The set of two isolated cell populations of claim 15 wherein the disease state is a genetic disease.

19. The set of two isolated cell populations of claim 15 wherein the disease state is Down's syndrome, Huntington's disease, or Lesch-Nyhan disease.

20. The set of two isolated cell populations of claim 15, further comprising at least 60% pluripotent stem cell-derived extraembryonic endoderm-like cells.

21. The set of two isolated cell populations of claim 15, further comprising at least 90% pluripotent stem cell-derived extraembryonic endoderm-like cells.

22. The set of two isolated cell populations of claim 15 wherein the pluripotent stem cells are embryonic stem cells.

23. The set of two isolated cell populations of claim 15 wherein the pluripotent stem cells are human pluripotent stem cells.

24. The set of two isolated cell populations of claim 23 wherein the pluripotent stem cells are human embryonic stem cells.

25. The set of two isolated cell populations of claim 15 wherein the first population is one primate pluripotent stem cell.

26. The set of two isolated cell populations of claim 15 wherein the one or more pluripotent stem cells are derived from inner cell mass cells.

27. The set of two isolated cell populations of claim 15, wherein the extraembryonic endoderm-like cells express two or more of the following: GATA6, DAB-2, basement membrane genes, laminin (LAMC1), collagens, fibronectin (FN1), or nidogens.

28. The set of two isolated cell populations of claim 15, wherein medium preconditioned by the extraembryonic endoderm-like cells causes proliferation of human embryonic stem cells without differentiation.

29. The set of two isolated cell populations of claim 15, wherein the second cell population has been obtained by culturing the pluripotent stem cells on an extracellular matrix on a solid substrate, and selecting cells having said criteria.

30. A culture system for maintaining undifferentiated growth of human embryonic stem cells comprising: a substrate covered with human embryonic stem cell-derived extraembryonic endoderm-like feeder cells and one or more undifferentiated human embryonic stem cells.

31. The culture system of claim 30, wherein at least 60% of the human embryonic stem cells remain substantially undifferentiated after 20 passages.

32. The culture system of claim 31, wherein at least 78% of the human embryonic stem cells remain substantially undifferentiated after 20 passages.

33. The culture system of claim 30 wherein the undifferentiated human embryonic stem cells derive from one human embryonic stem cell.

34. The culture system of claim 30 wherein the one or more undifferentiated human embryonic stem cells derive from inner cell mass cells.

35. A culture system for maintaining undifferentiated growth of human embryonic stem cells comprising: a medium preconditioned by extraembryonic endoderm-like feeder cells, and one or more undifferentiated human embryonic stem cells.

36. The culture system of claim 35, wherein at least 60% of the human embryonic stem cells remain substantially undifferentiated after 20 passages.

37. The culture system of claim 36, wherein at least 80% of the human embryonic stem cells remain substantially undifferentiated after 20 passages.

38. The culture system of claim 35 wherein the undifferentiated human embryonic stem cells derive from one human embryonic stem cell.

39. The culture system of claim 35 wherein the undifferentiated human embryonic stem cells derive from inner cell mass cells.

40. A method for culturing undifferentiated mammalian cells comprising, obtaining a single undifferentiated mammalian embryonic stem cell, and inoculating the single cell onto mammalian extraembryonic endoderm-like feeder cells in a nutrient medium.

41. The method of claim 40, wherein the mammalian embryonic stem cells are human, primate, mouse, or rat.

42. The method of claim 40, wherein the mammalian extraembryonic endoderm-like feeder cells are human, primate, mouse, or rat.

43. The method of claim 40, wherein the human embryonic stem cells are WA09 human embryonic stem cell line.

44. The method of claim 40, wherein the extraembryonic endoderm-like feeder cells are positive for cell markers of one or more of the following: GATA6, DAB-2, basement membrane genes, laminin (LAMC1), collagens, fibronectin (FN1), or nidogens.

45. The method of claim 40, further comprising obtaining a single undifferentiated human embryonic stem cell which comprises selecting a group of undifferentiated cells from a cell culture, and dissociating the group of undifferentiated cells into single cells.

46. The method of claim 45, wherein the dissociation method is enzymatic degradation.

47. The method of claim 46, wherein the enzymatic degradation is collagenase degradation.

48. The method of claim 40, wherein the method is used for establishing a clonal human embryonic stem cell line.

49. The method of claim 40, wherein the method is suitable for gene transfection.

50. A method for generating isolated primate extraembryonic endoderm-like cells comprising,
growing primate embryonic stem cells on extracellular matrix under feeder cell free conditions;
identifying extraembryonic endoderm-like cells as positive for cell markers of one or more of the following: GATA6, DAB-2, basement membrane genes, laminin (LAMC1), collagens, fibronectin (FN1), or nidogens; and
isolating extraembryonic endoderm-like cells from the embryonic stem cells.

51. The method of claim 50, wherein the primate embryonic stem cells are human embryonic stem cells.

52. The method of claim 50, further comprising isolating extraembryonic endoderm-like cells from the embryonic stem cells by mechanical dissection.

53. The method of claim 50, further comprising isolating extraembryonic endoderm-like cells from the embryonic stem cells by enzymatic digestion.


SEQUENCE LISTING (28 sequences)

SEQ ID NO: 1 - DNA, 19 bp, Artificial Sequence
  Oligonucleotide Primer
cttgaagaagagacccgat

SEQ ID NO: 2 - DNA, 19 bp, Artificial Sequence
  Oligonucleotide Primer
cttctgcacgctccactac

SEQ ID NO: 3 - DNA, 25 bp, Artificial Sequence
  Oligonucleotide Primer
acacgggagtgcatctactacaacg

SEQ ID NO: 4 - DNA, 25 bp, Artificial Sequence
  Oligonucleotide Primer
ttcatgagctgggccttccagacac

SEQ ID NO: 5 - DNA, 20 bp, Artificial Sequence
  Oligonucleotide Primer
agaacctgtcacaagctgtg

SEQ ID NO: 6 - DNA, 20 bp, Artificial Sequence
  Oligonucleotide Primer
cacagcaagctgaggatgtc

SEQ ID NO: 7 - DNA, 26 bp, Artificial Sequence
  Oligonucleotide Primer
tggcaccacacctttctacaatgagc

SEQ ID NO: 8 - DNA, 25 bp, Artificial Sequence
  Oligonucleotide Primer
gcacagcttctccttaatgtcacgc

SEQ ID NO: 9 - DNA, 21 bp, Artificial Sequence
  Oligonucleotide Primer
gaacctgtgcgagtggatgcg

SEQ ID NO: 10 - DNA, 22 bp, Artificial Sequence
  Oligonucleotide Primer
ggtctatggctgtgggtgggag

SEQ ID NO: 11 - DNA, 20 bp, Artificial Sequence
  Oligonucleotide Primer
ctcttaccttaccatcgacc

SEQ ID NO: 12 - DNA, 20 bp, Artificial Sequence
  Oligonucleotide Primer
ctccagagcatggtacatgg

SEQ ID NO: 13 - DNA, 20 bp, Artificial Sequence
  Oligonucleotide Primer
catcaagacggagcctggcc

SEQ ID NO: 14 - DNA, 20 bp, Artificial Sequence
  Oligonucleotide Primer
tgactgtcggccaagaccag

SEQ ID NO: 15 - DNA, 20 bp, Artificial Sequence
  Oligonucleotide Primer
gcttggttctcgttgagtgg

SEQ ID NO: 16 - DNA, 23 bp, Artificial Sequence
  Oligonucleotide Primer
caggagcttataggggctcagac

SEQ ID NO: 17 - DNA, 20 bp, Artificial Sequence
  Oligonucleotide Primer
agtaagctgcacatggaagg

SEQ ID NO: 18 - DNA, 20 bp, Artificial Sequence
  Oligonucleotide Primer
attgtggctcaattctgtgc

SEQ ID NO: 19 - DNA, 20 bp, Artificial Sequence
  Oligonucleotide Primer
ccgcatgtacaacatgatgg

SEQ ID NO: 20 - DNA, 20 bp, Artificial Sequence
  Oligonucleotide Primer
cttcttcatgagcgtcttgg

SEQ ID NO: 21 - DNA, 20 bp, Artificial Sequence
  Oligonucleotide Primer
ccatgactccaacttccacc

SEQ ID NO: 22 - DNA, 20 bp, Artificial Sequence
  Oligonucleotide Primer
acggaggacgtgacttcggc

SEQ ID NO: 23 - DNA, 20 bp, Artificial Sequence
  Oligonucleotide Primer
ggcaaacaacccacttctgc

SEQ ID NO: 24 - DNA, 20 bp, Artificial Sequence
  Oligonucleotide Primer
tgttccaggcctgattgttc

SEQ ID NO: 25 - DNA, 25 bp, Artificial Sequence
  Oligonucleotide Primer
cgtgaagctggagaaggagaagctg

SEQ ID NO: 26 - DNA, 25 bp, Artificial Sequence
  Oligonucleotide Primer
caagggccgcagcttacacatgttc

SEQ ID NO: 27 - DNA, 20 bp, Artificial Sequence
  Oligonucleotide Primer
cgcacggaatttgaacagta

SEQ ID NO: 28 - DNA, 20 bp, Artificial Sequence
  Oligonucleotide Primer
ggatcagggacctgtcacac